(19) United States
(12) Patent Application Publication
(10) Pub. No.: US 2008/0118572 A1
(43) Pub. Date: May 22, 2008

(54) METHODS AND COMPOSITIONS FOR REDUCING THE RISK OF ADVERSE CARDIOVASCULAR EVENTS ASSOCIATED WITH THE ADMINISTRATION OF ARTIFICIAL BLOOD

(76) Inventor: Harold Richard Hellstrom, Fayetteville, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/869,433

(22) Filed: Oct. 9, 2007

Related U.S. Application Data
(60) Provisional application No. 60/850,792, filed on Oct. 10, 2006.

(51) Int. Cl. A61K 35/14; A61P 9/00

(52) U.S. Cl. 424/529

(57) ABSTRACT

Methods and compositions are provided for reducing the risk of adverse cardiovascular (CV) events associated with the administration of artificial blood. The methods involve the administration of a composition comprising artificial blood and one or more preventative pharmaceutical agents for reducing the risk of adverse CV events. In specific embodiments, an agent is selected from the group consisting of hydroxymethylglutaryl-coenzyme A reductase inhibitors (statins), angiotensin converting enzyme (ACE) inhibitors, aspirin, corticosteroids, PPAR agents, and vasodilators. Kits for administering artificial blood together with one or more pharmaceutical agents that reduce the risk of adverse CV events are also provided.

### CROSS-REFERENCE TO RELATED APPLICATIONS

This application claims the benefit under 35 U.S.C. § 119 of U.S. Provisional Application No. 60/850,792 filed Oct. 10, 2006, the contents of which is incorporated by reference herein in its entirety.

### FIELD OF THE INVENTION

The present invention involves methods and compositions for administering and co-administering pharmaceutical agents that reduce the risk of myocardial infarction and/or other adverse cardiovascular (CV) events associated with the administration of artificial blood. Kits for administering artificial blood together with pharmaceutical agents that reduce the risk of adverse CV events are also provided.

### BACKGROUND OF THE INVENTION

Many individuals are at an elevated risk of suffering serious to life-threatening CV events, such as myocardial infarction (heart attack), cardiac arrest, congestive heart failure, stroke, peripheral vascular disease and/or claudication. The risk factors are numerous and widespread throughout the world population. They include diabetes, hypercholesterolemia (high serum cholesterol), hypertension, angina, systemic lupus erythematosus, cigarette smoking, prior heart attacks or strokes, hemodialysis, hyperhomocysteine levels, obesity, sedentary lifestyle, and others. Recently, there have been a number of reports associating the administration of artificial blood with an elevated risk of serious adverse CV events. Thus, the administration of artificial blood is considered an additional risk factor that can lead to serious and life threatening CV problems.

### Artificial Blood

Artificial blood products have great potential for use in surgical and trauma patient situations. A pharmaceutically acceptable artificial blood product is particularly useful in surgical situations where donor blood is not readily available and in combat situations in lieu of saline. Saline is typically administered to trauma patients in combat due to the unavailability of blood. If used instead of saline, the oxygen-carrying capacity of artificial blood could reduce the death rate of trauma patients by 25% by some estimates. Artificial blood products are currently under review by the U.S. Food and Drug Administration. Previous clinical trials have indicated that there is a risk of increased adverse events from artificial blood products in comparison with donor blood. Such risks as stroke, cardiac arrest and hypertension, have lead to careful scrutiny of artificial blood products.

Methods of reducing the risk of adverse cardiovascular events associated with the administration of pharmaceutical agents which favor adverse CV events have been previously described by Hellstrom, US 2007/0037797 published Feb. 15, 2007, the contents of which is incorporated by reference herein in its entirety.

Thus, there remains a need for a method of administering artificial blood while reducing the risk of adverse CV events associated therewith. It is an object of the present invention to reduce the risk of adverse CV events associated with the administration of artificial blood, by the administration or co-administration of one or more pharmaceutical agents for reducing the risk of adverse CV events.

### SUMMARY OF THE INVENTION

In accordance with the present invention, the CV risks associated with the administration of artificial blood can be reduced by the administration of the artificial blood with one or more preventative pharmaceutical agents for reducing the risk of adverse CV events. In some embodiments, the one or more preventative pharmaceutical agents can be selected from the group consisting of hydroxymethylglutaryl-coenzyme A reductase inhibitors (statins) and angiotensin converting enzyme (ACE) inhibitors. Further, the one or more preventative pharmaceutical agents may be selected from the group consisting of aspirin, corticosteroids, PPAR agents, and vasodilators.

The present invention also provides a program for reducing the risk of adverse CV events associated with the administration artificial blood that involves administering the one or more preventative pharmaceutical agents for a period prior to the administration of artificial blood. Further, the one or more preventative pharmaceutical agents may be administered for an additional period of time after the administration of artificial blood is completed, i.e., for at least a week.

The present invention is directed to methods for reducing the risk of an adverse CV event in a subject to be treated with artificial blood, the method comprising administering:
(a) a therapeutically effective amount of artificial blood; and
(b) one or more preventative pharmaceutical agents in an amount effective to reduce the risk of one or more adverse CV events.

The present invention also provides a method for reducing the risk of one or more adverse CV events in a subject to be treated with artificial blood, the method comprising co-administering:
(a) a therapeutically effective amount of artificial blood; and
(b) one or more preventative pharmaceutical agents in an amount effective to reduce the risk of one or more adverse CV events,
   wherein the co-administration may be parenterally as a unit dosage form. Further, the labeling may indicate the presence of a preventative pharmaceutical agent with the artificial blood.

The present invention additionally provides a kit for reducing the risk of one or more adverse CV events in a subject to be treated with artificial blood, comprising:
(a) a therapeutically effective amount of artificial blood; and
(b) instructions for administering one or more preventative pharmaceutical agents in an amount effective to reduce the risk of one or more adverse CV events.

An additional aspect of the invention is directed to compositions comprising:
(a) a therapeutically effective amount of artificial blood; and
(b) one or more preventative pharmaceutical agents in an amount effective to reduce the risk of at least one adverse CV event associated with administration of artificial blood to a subject.

The present invention thus addresses the need to, inter alia, provide a benefit of artificial blood, i.e., to treat or prevent hypoxia due to blood loss, anemia or shock, to a subject in need thereof, while simultaneously reducing the risk of one or more adverse CV events that are associated with the administration of artificial blood.

### DETAILED DESCRIPTION

The present invention advantageously provides methods, compositions, and kits for reducing the risk of an adverse CV event associated with the administration of artificial blood. Risk factor reduction, that is, eliminating risk factors associated with adverse CV events is the standard of care in medicine at the present time. However, in the case of artificial blood, administration may be required to treat or prevent hypoxia due to blood loss, anemia or shock. Thus, risk factor reduction is an undesirable or ineffective way to reduce the incidence of adverse CV events inherently associated with artificial blood administration. It has now been discovered that risk factor reduction is not the only way to reduce the risk of adverse CV events.

For example, the insights gained from studying years of literature reports have resulted in the discovery that combining administration of artificial blood with one or more pharmaceutical agents suitable for reducing the risk of an adverse CV event surprisingly will reduce the risk of the adverse CV event associated with artificial blood administration. In a particular embodiment, the invention is based on the discovery that administering an artificial blood product, such as POLYHEME® or HEMOPURE®, with a statin, such as LIPITOR®, and/or an ACE inhibitor, such as ALTACE®, permits treatment or prevention of hypoxia with reduced risk of developing an adverse CV event. The addition of more than one preventative agent may further reduce the risk of an adverse CV event. For example, aspirin may be administered with a statin and/or an ACE inhibitor if excessive bleeding is not a factor in the subject being treated.

### DEFINITIONS

The term “adverse cardiovascular (CV) event,” or simply “cardiovascular (CV) event,” as used herein refers, generally, to a disorder or disease of the cardiovascular system resulting from progressive vascular damage. Although the event may have a rather sudden onset, it can also refer to a progressive worsening of such a disorder or disease. Examples of adverse CV events include, without limitation: claudication, hypertension, cardiac arrest, myocardial infarction, ischemia, stroke, transient ischemic attacks, worsening of angina, congestive heart failure, left ventricular hypertrophy, sudden cardiac death, arrhythmias, thromboembolism and arterial and venous thromboses. Examples of progressive vascular diseases are those that affect the cerebral, coronary, renal, or peripheral circulations.

The terms “artificial blood,” “artificial blood product,” “artificial hemoglobin,” and “blood substitute” are synonymous and refer to hemoglobin based blood substitutes. The hemoglobin based blood substitute may be derived from human or bovine hemoglobin. Examples of artificial blood products being investigated in the United States are POLYHEME®, manufactured by Northfield Laboratories, Inc., and HEMOPURE®, manufactured by Biopure Corporation.

The term “administration” generally refers to providing a subject with an appropriate dose of a treatment regimen.

The term “co-administration” as used herein refers to the administration of artificial blood and one or more preventative pharmaceutical agents at approximately the same time. For example, co-administration refers to providing a subject with an appropriate dose of artificial blood and one or more preventative pharmaceutical agents within a forty-eight hour time period of one another. Additional examples of co-administration provide artificial blood and at least one preventative pharmaceutical agent within a twenty-four hour time period, a twelve hour time period, a six hour time period, or a three hour time period, of one another. An additional example of co-administration is administering to a subject a unit dosage form containing both artificial blood and at least one preventative pharmaceutical agent.

By “pharmaceutically acceptable,” such as in the recitation of a “pharmaceutically acceptable carrier,” is meant the conventional definition as understood by one skilled in the art of pharmaceutical sciences. For example, a “pharmaceutically acceptable” material is generally not biologically or otherwise undesirable when administered to a subject, i.e., the material may be incorporated into a pharmaceutical composition to be administered to the subject without causing undesirable biological effects or interacting in a deleterious manner with any of the other components of the composition in which it is contained.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

The terms “effective amount” and “therapeutically effective amount,” in reference to artificial blood, a preventative pharmaceutical agent, a drug or other pharmacologically active agent, are synonymous and refer to a nontoxic but sufficient amount of the drug or agent to provide the desired prophylactic or therapeutic effect. In the combination therapy of the present invention, an “effective amount” of one component of the combination is the amount of that component that is effective to provide the desired effect when used in combination with the other components of the combination. The amount that is “effective” will vary from subject to subject, depending on the age and general condition of the individual, the particular active agent or agents employed, and the like. An appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

The terms “cardiovascular (CV) risk,” “elevated cardiovascular (CV) risk” and “cardiovascular (CV) risk factor” as used herein refer to an increased risk of incurring an adverse CV event, peripheral vascular disease, coronary heart disease, restenosis, or atherosclerosis. In a given individual, such risk may be due to the administration of artificial blood. The conditions or factors that lead to elevated CV risk include, without limitation: administration of artificial blood itself, and standard risk factors, such as, systemic lupus erythematosus, current or prior cigarette smoking, diabetes, hypertension, stroke, angina, hemodialysis, organ transplant, manifest coronary artery disease, history of myocardial infarction, history of transient ischemic attacks or stroke, history of peripheral vascular disease, angina, hypertension, hypercholesterolemia, obesity, atherosclerosis, kidney disease, Chlamydia infection, Bartonella infection, and obstructive pulmonary disease.

The term “overall risk” for an adverse CV event, as used herein, refers to the total risk of an adverse CV event in a patient, said total risk being associated with administration of artificial blood, and/or one or more standard risk factors as set forth herein. Thus the overall risk is a risk due to the combination of the risk associated with artificial blood administration and the risks associated with standard risk factors. The overall risk for an adverse CV event can be lowered through the practice of the methods of the present invention through administration of a pharmaceutical agent for lowering the risk of an adverse CV event.

“Reducing the risk” or “reduction of risk” of occurrence of adverse CV events refers to lowering the overall risk of occurrence of any of the conditions or factors set forth herein, in a patient at risk to developing the conditions as a result of, or exacerbated by, the administration of artificial blood.

The terms “pharmaceutical agent for reducing the risk of cardiovascular (CV) event(s)” and “preventative pharmaceutical agent” as employed herein, are synonymous, and refer to any pharmaceutical agent that is known in the art to reduce the risk of the CV events set forth herein. Pharmaceutical agents for reducing the risk of CV event(s) include aspirin, statins, ACE inhibitors, corticosteroids, peroxisome proliferator-activated receptor (PPAR) agents, and vasodilators.

The term “adverse CV event risk reducing amount of one or more preventative pharmaceutical agents,” as used herein, refers to an amount of the preventative agent sufficient to counteract (e.g., significantly lower) at least one risk of an adverse CV event associated with artificial blood.

### The Risk/Prevention Balance and Altered Homeostatic Theory

The current and standard methods for preventing ischemic heart disease (IHD), hypertension, and other adverse CV events are based solely on removal or reduction of risk factors. As evidence, risk factors for IHD are separated into two groups, modifiable and unmodifiable. Modifiable risk factors are hypertension, smoking, diabetes, obesity, physical activity, and atherogenic diet. Non-modifiable risk factors are age, male sex, and family history of premature IHD (Expert Panel on Detection, Evaluation, and Treatment of High Blood Cholesterol in Adults. Detection, evaluation, and treatment of high blood cholesterol in adults (Adult Treatment Panel III): third report of the National Cholesterol Education Program (NCEP) Expert Panel. Circulation 2002; 106:3145-3421). As might be expected, discussion of risk factors in this report was limited to the modifiable group, and the apparent impossibility of modifying the unmodifiable risk factor of age no doubt has limited interest in preventing disease in older individuals.

The prevention of, for example, IHD solely by risk factor reduction (i.e., the standard method) is likely based on the premise that multiple risk factors for IHD operate by individual mechanisms. It might seem reasonable that diverse risk factors as dyslipidemia, emotional stress, cold exposure, and hypertension have individual mechanisms; if each risk factor has its own mechanism, the only way to reduce the risk of IHD is to remove individual risk factors.

In contrast to this apparent standard view for the prevention of CV events is the prevention of CV events based on the principle of favorably altering the total balance of risk and preventative factors—a risk/prevention balance. Without wishing to be bound by theory, the principle of improving the overall risk/prevention balance is based on the principle that risk factors operate by a single (if complex) mechanism and are opposed by a single (if complex) mechanism of preventative factors. Also, it is asserted that prevention of disease based on favorably altering the overall risk/prevention balance more accurately reflects basic mechanisms of, for example, IHD and hypertension; if so, prevention based on this approach should be more effective.

This hypothesis first asserted that IHD and hypertension have a common basic mechanism of altered homeostasis (See, Hellstrom H R. Med. Hypotheses. 1999; 53:194-199; Hellstrom H R. Med. Hypotheses. 2003; 60:12-25; Hellstrom H R. Med. Hypotheses. In press.) and now is also applied to diabetes. The hypothesis avers that risk factors shift homeostasis toward disease, and that preventative factors shift homeostasis toward health; more specifically, risk factors cause inappropriate acute and/or chronic activation of sympathetic defensive action (fight/flight), and preventative factors shift homeostasis beneficially toward parasympathetic rest (conservation/withdrawal). The autonomic nervous system involves basic functions including vascular, nervous system, and metabolic function, and shifts of homeostasis are considered to include major mechanisms of these systems (Lefkowitz R J, Hoffman B B, Taylor P. “Neurotransmission: the autonomic and somatic motor nervous systems.” In: Hardman J G, Limbird L E, Molinoff P B, Ruddon R W, Gilman A G, editors. The Pharmacological Basis of Therapeutics. New York: McGraw-Hill, 1996: 105-139). Thus, without wishing to be bound by theory, if risk factors operate by sympathetic activation and preventative factors by parasympathetic activation, then risk and preventative factors operate by a single (if complex) opposing mechanism.

The position that risk and preventative factors operate by a single (if complex) opposing mechanism carries with it a major implication. If risk factors operate by a single mechanism and are opposed by preventative factors operating through an opposing mechanism, any preventative factor can oppose any risk factor and favorably alter the risk/prevention balance; as any risk factor can be opposed, this position allows for attenuation of unmodifiable risk factors such as age. This position also allows for attenuation of risk factors associated with the administration of artificial blood by administering one or more preventative pharmaceutical agents.

The view that inappropriate homeostatic shifts causing disease involve sympathetic activation, and that beneficial shifts involve parasympathetic activation, can be used to support the idea of a risk/prevention balance. The autonomic nervous system is known to operate through balance of sympathetic and parasympathetic activity (Lefkowitz R J, et al. In: The Pharmacological Basis of Therapeutics. New York: McGraw-Hill, 1996: 105-139) and a like balance is proposed for risk and preventative factors, i.e., a risk/prevention balance.

The theory has major similarities to other proposals. Favorably, sympathetic activation has been implicated in IHD, hypertension, and diabetes, and common mechanisms for these disorders have been proposed. However, the hypothesis underlying the present invention appears unique in its position that risk and preventative factors operate in a dynamic balance though shifts of homeostasis.

The hypothesis can be regarded as a modification and expansion of previous proposals. Both Selye's stress syndrome (Selye H. The Stress of Life: Revised Edition. New York: McGraw-Hill Book Co, 1978 and McEwen B S. N Engl J. Med. 1998; 338:171-179) and the altered homeostatic theory give a basic role to sympathetic hyperactivity and have been applied to IHD, hypertension, and diabetes. Also, IHD (Remme W J. Eur Heart J. 1998; 19 (Suppl F):F62-F71) and hypertension (Julius S. Clin Exp Hypertens. 1995; 17:375-386) have been attributed to inappropriate activation of the sympathetic nervous system and to activation of fight/flight, and diabetes has been attributed to sympathetic activation (M, Rumantir M, Wiesner G et al. Am J. Hypertens. 2001; 14:304S-309S) and to stress induced disturbance of the hypothalamic-pituitary-adrenal axis—which includes sympathetic activation (Rosmond R. Med Sci Monit. 2003; 9:RA35-RA39).

The altered homeostatic theory also is similar to proposals of common pathogenesis involving IHD, hypertension, and/or diabetes. As examples, IHD and diabetes have been related to inflammation (Ridker P M. Circulation. 2002; 105:2-4 and Pickup J C. Diabetes Care. 2004; 27:813-823) hypertension and IHD have been ascribed to the defense reaction (Julius S. Clin Exp Hypertens. 1995; 17:375-386), and the metabolic syndrome with its clustering of IHD, hypertension, diabetes, dyslipidemia, a thrombotic tendency, and obesity has been attributed to sympathetic activation and insulin resistance (Reaven G M, Lithell H, Landsberg L. N Engl J. Med. 1996; 334:374-381).

Table 1, below, summarizes the associations of attributes of sympathetic fight/flight with risk factors for IHD, hypertension, and diabetes. The superscripts in the table reference the publications, listed at the end of the specification, in which the associations between sympathetic fight/flight and risk factors have been reported for each particular entry.

As evidenced by the data represented in Table 1, risk factors are considered to operate by a single mechanism involving sympathetic activation. Twelve separate and diverse risk factors for IHD, hypertension, and diabetes, most of which are major, express sympathetic activation, and this provides evidence that risk factors operate by the single mechanism which involves sympathetic activation.

There is further evidence that risk factors are involved with sympathetic activation. Namely, major attributes of sympathetic activation are equivalent to four major risk factors for IHD, hypertension, and diabetes. As indicated in Table 1, sympathetic fight/flight prompts endothelial dysfunction (a thrombotic and vasoconstrictive tendency See, Verma S, Anderson T J. Circulation. 2002; 105:546-549), and increases levels of lipids, inflammation, and glucose. These four attributes of sympathetic fight/flight are equivalent to the risk factors for IHD, hypertension, and diabetes of endothelial dysfunction, dyslipidemia, inflammation, and insulin resistance (Table 1).

Without wishing to be bound by any particular theory, it is assumed that defensive sympathetic activation, when inappropriate and prolonged, can cause disease. In an acute fight/flight situation, activating the sympathetic nervous system and its associated components permits a more effective defense and can be lifesaving. Raising levels of lipids and glucose fuels the defense; activating a tendency toward thrombosis and vasoconstriction protects against exsanguination after injury (i.e., the hemostatic response), raising blood pressure (i.e., vasoconstriction) and heart rate enhances response capabilities, and activating inflammation protects against injury-induced infections. However, inappropriate and prolonged sympathetic activation is considered to change these helpful components of sympathetic activation to sympathetic-related risk factors of dyslipidemia, insulin resistance, endothelial dysfunction, and inflammation.

The sympathetic nervous system is highly interrelated, and reducing one of these risk factors/attributes of sympathetic activation, for example, reducing cholesterol values, would result in reduction of other risk factors and foster a shift from sympathetic activation to beneficial parasympathetic activation. Also, through parasympathetic activation, improving dyslipidemia, for example, will also have the helpful action of improving other sympathetic risk factors as inflammation, endothelial dysfunction, and glucose intolerance.

TABLE 1
Associations of Risk Factors with IHD, Hypertension, and Diabetes, and with Sympathetic Activation

| | | | | Sympathetic | | Attributes of Sympathetic Activation | | | | |
|---|---|---|---|---|---|---|---|---|---|---|
| | | | | Activation | | (Which also are Major Risk Factors | | | | |
| | Disorders | | | ↑ Sym- | ↓ Parasym- | | | ↑ Lipids/ | | ↑ Glucose/ |
| | | Hyper- | | pathetic | pathetic | Endothelial | Thrombotic | Dyslip- | Inflam- | Insulin |
| | IHD | tension | Diabetes | Activity | activity | dysfunction* | tendency | idemia | mation | resistance |
| Sympathetic | | | | +¹ | +¹ | +² | +³ | +⁴ | +⁴ | +‡³ |
| Fight/Flight & its | | | | | | | | | | |
| Attributes | | | | | | | | | | |
| Sympathetic | +⁵ | +⁶ | +⁷ | | | | | | | |
| Hyperactivity | | | | | | | | | | |

“Sympathetic” Risk Factors

| | | | | | | | | | | |
|---|---|---|---|---|---|---|---|---|---|---|
| Endothelial | +⁸ | +⁹ | +⁸ | +² | — | | +⁸ | +⁸ | +⁸ | +⁸ |
| dysfunction* | | | | | | | | | | |
| Dyslipidemia | +¹⁰ | +¹¹ | +¹⁰ | +⁴ | +¹² | +⁸ | +¹³ | | +¹⁴ | +¹⁵ |
| Inflammation | +¹⁶ | +¹⁷ | +¹⁸ | +⁴ | +‡4 | +⁸ | +¹⁹ | +²⁰ | | +²¹ |
| Insulin Resistance/ | +¹⁰ | +¹¹ | | +²² | +²³ | +⁸ | +²⁴ | +¹⁵ | +¹⁸ | |
| diabetes | | | | | | | | | | |

Other Risk Factors

| | | | | | | | | | | |
|---|---|---|---|---|---|---|---|---|---|---|
| Aging | +¹⁰ | +¹¹ | +²⁵ | +²⁶ | +²⁷ | +⁸ | +²⁸ | +²⁹ | +³⁰ | +³¹ |
| Obesity | +¹⁰ | +³² | +³³ | +³⁴ | +³⁵ | +⁸ | +³³ | +³³ | +³⁶ | +³³ |
| Unexercised State | +¹⁰ | +³⁷ | +³⁸ | +³⁹ | +⁴⁰ | +⁸ | +⁴¹ | +⁴² | +⁴² | +⁴³ |
| Stress | +⁴⁴ | +⁴⁵ | +⁴⁶ | +⁴⁷ | +⁴⁸ | +⁴⁴ | +⁴⁴ | +⁴ | +⁴ | +⁴⁹ |
| ↑ Blood Pressure | +⁴⁴ | +⁴⁵ | +⁴⁶ | +⁴⁷ | +⁴⁸ | +⁴⁴ | +⁴⁴ | +⁴ | +⁴ | +⁴⁹ |
| Hypertension | | | | | | | | | | |
| Smoking | +¹⁰ | +¹¹ | +⁵⁰ | +⁵¹ | +‡⁵¹ | +⁸ | +⁵¹ | +⁵² | +¹⁸ | +⁵³ |
| Circadian Rhythm | +⁵⁴ | +⁵⁴ | +§⁵⁵ | +⁵⁴ | +‡⁵⁴ | +‡⁵⁴ | +⁵⁴ | +⁵⁶ | +⁵⁷ | +§⁵⁵ |
| Cold | +⁵⁸ | +⁵⁹ | +⁶⁰ | +⁶¹ | +‡⁶¹ | +‡⁶¹ | +⁶¹ | +⁶² | +⁶³ | +⁶⁴ |

*Expresses a tendency toward thrombosis and vasoconstriction
†↑ glucose levels only for sympathetic activation
‡By inference, as opposite agent causes opposite effect
§Plasma glucose levels
—No Information Found

Table 2 summarizes the beneficial effects of pharmaceutical agents on CV related diseases, on risk factors for adverse CV events, and on other situations not directly related to CV events. The superscripts in the table reference the publications, listed at the end of the specification, in which the effects of pharmaceutical agents on adverse CV related diseases have been reported for each particular entry.

TABLE 2
Beneficial Effects of Pharmaceutical Agents on Diseases,
on Parasympathetic Activation, and on Other Situations

| Pharmaceutical Agents | | | | |
|---|---|---|---|---|
| | | Anti- | | |
| | | Angio- | PPAR* | Estrogen/ |
| Statins | Aspirin | tension | agonists | Progesterone |

Diseases

| | | | | | |
|---|---|---|---|---|---|
| Prevent IHD | +¹⁰ | +†⁶⁵ | +⁶⁶ | +⁶⁷ | cr⁶⁸ |
| Treat IHD | +⁶⁹ | +⁷⁰ | +⁶⁶ | +⁷¹ | — |
| Prevent Hypertension | +§⁷² | — | +‡⁷³ | +⁷⁴ | +⁷⁵ |
| Treat Hypertension | +§⁷⁶ | +§†⁷⁷ | +¹¹ | — | — |
| Prevent Diabetes | +⁷⁸ | — | +⁷⁹ | +⁸⁰ | +⁸¹ |
| Treat Diabetes | +⁸² | — | +⁸³ | +⁸⁴ | — |

Parasympathetic Activation and Improvement of
Sympathetic activation, i.e. Major Risk Factors

| | | | | | |
|---|---|---|---|---|---|
| ↑ Parasympath. Activity | +¹² | +⁸⁵ | +⁸⁶ | — | +⁸⁷ |
| ↓ Sympathetic activity | +⁸⁸ | — | +⁷⁹ | +⁸⁹ | +⁹⁰ |
| ↓ Endothelial dysfunction | +⁸ | +⁹¹ | +⁸ | +⁹² | +⁸ |
| ↓ Thrombosis | +⁹³ | +†⁹⁴ | +⁹⁵ | +⁹² | x⁹⁶ |
| ↓ Dyslipidemia | +¹⁰ | +⁹⁷ | +⁹⁸ | +⁹⁹ | +¹⁰⁰ |
| ↓ Inflammation | +¹⁰¹ | +†⁹⁴ | +¹⁰² | +⁹² | +¹⁰³ |
| ↓ Insulin Resistance | +⁸² | +⁹⁷ | +⁷⁹ | +⁸⁴ | +¹⁰⁴ |

Other Situations/Disorders

| | | | | | |
|---|---|---|---|---|---|
| ↑ Cognitive function | +¹⁰⁵ | +¹⁰⁶ | +¹⁰⁷ | — | +¹⁰⁸ |
| ↑ Bone density | +¹⁰⁹ | +¶¶¹¹⁰ | — | cr¹¹¹ | +¹¹² |
| ↓ Alzheimer's disease | +cr¹¹³ | +†¹¹⁴ | +¹¹⁴ | +‡¹¹⁵ | +¹¹⁶ |
| ↓ Atrial fibrillation | +¹¹⁷ | — | +¹¹⁸ | — | — |
| ↓ Cancer | +¹¹⁹ | +¹²⁰ | +¹²¹ | cr¹²² | x¹¹² |

+Positive association
crConflicting results
—No information found
na No association
xNegative association
*Peroxisome proliferator-activated receptor
†Low dose aspirin
‡Proposed
§Known to lower blood pressure
¶By inference, as opposite agent causes opposite action
#Possibility raised
**Further studies needed
††Study of fructose-fed rats
§§Study of growing rabbits
¶¶Plus COX-2 inhibitor

Table 3 summarizes the beneficial effects of certain “lifestyle agents”, for example healthy living habits (e.g., maintaining proper weight, exercise, stress reduction, and healthy dietary habits) on CV related diseases, on risk factors for adverse CV events, and on other situations not directly related to adverse CV events. The superscripts in the table reference the publications, listed at the end of the specification, in which the effects of lifestyle agents on CV related diseases have been reported for each particular entry.

TABLE 3
Beneficial Effects of Lifestyle Agents on Diseases, on Parasympathetic Activation, and on Other Situations

| Lifestyle Agents | | | | | | | |
|---|---|---|---|---|---|---|---|
| | | | | | | | Diet high |
| | | | Moderate | Fish oil/ | | Mediterranean | in |
| Exercise | ↓ Weight | ↓ Stress | alcohol | Fish | Nuts | diet | flavonoids |

Diseases

| | | | | | | | | |
|---|---|---|---|---|---|---|---|---|
| Prevent IHD | +¹²³ | +¹⁰ | +⁴⁴ | +¹²⁴ | +¹²⁵ | +¹²⁶ | +¹²⁷ | +¹²⁸ |
| Treat IHD | +¹²³ | — | +⁴⁴ | +¹²⁴ | +¹²⁹ | — | +¹²⁷ | — |
| Prevent Hypertension | +¹³⁰ | +¹³⁰ | +¶¹³¹ | x¹³⁰ | +¹²⁵ | — | +§¹³² | +¹³³ |
| Treat Hypertension | +¹³⁰ | +¹³⁰ | +¹³⁴ | x¹³⁰ | +¹³⁵ | — | +§¹³² | — |
| Prevent Diabetes | +⁴² | +¹³⁶ | +¶#¹³⁷ | +¹³⁸ | — | +¹³⁹ | +¹⁴⁰ | — |
| Treat Diabetes | +⁴² | +¹³⁶ | +¶⁴¹ | +**¹³⁸ | — | — | — | — |

Parasympathetic Activation and Improvement of
Sympathetic activation, i.e. Major Risk Factors

| | | | | | | | | |
|---|---|---|---|---|---|---|---|---|
| ↑ Parasympath. | +¹⁴² | +¹⁴³ | +¹⁴⁴ | x¹⁴⁵ | +¹⁴⁶ | — | — | — |
| Activity | | | | | | | | |
| ↓ Sympathetic activity | +³⁹ | +¹⁴⁷ | +¹⁴⁸ | x¹⁴⁹ | +¹⁵⁰ | — | — | — |
| ↓ Endothelial | +¹⁵¹ | +¹⁵² | +¶¹⁵³ | +¹⁵⁴ | +¹⁴⁶ | +¹²⁶ | +¹⁵⁵ | +¹²⁸ |
| dysfunction | | | | | | | | |
| ↓ Thrombosis | +⁴¹ | +⁴¹ | +¶⁴¹ | +¹⁵⁶ | +¹⁴⁶ | — | — | +¹⁵⁷ |
| ↓ Dyslipidemia | +⁴² | +¹⁵⁸ | +¹³⁴ | +¹⁵⁹ | +¹⁴⁶ | +¹⁶⁰ | na¹²⁷ | +¹⁶¹ |
| ↓ Inflammation | +¹⁶² | +¹⁵² | +¹⁶³ | +¹⁶⁴ | +¹⁴⁶ | — | +¹⁵⁵ | +¹²⁸ |
| ↓ Insulin Resistance | +⁴² | +¹⁵⁸ | +¶¹⁶⁵ | +¹⁰⁴ | cr¹⁶⁶ | +**¹⁶⁷ | +¹⁵⁵ | na††¹³³ |

Other Situations/Disorders

| | | | | | | | | |
|---|---|---|---|---|---|---|---|---|
| ↑ Cognitive function | +¹⁶⁸ | x¹⁶⁹ | +¶¹⁰⁸ | +¹⁷⁰ | +¹⁷¹ | — | — | +¹⁷² |
| ↑ Bone density | +¹⁷³ | x¹⁷⁴ | — | +¹⁷⁵ | x§§¹⁷⁶ | — | — | +¹⁶¹ |
| ↓ Alzheimer's disease | +¹⁷⁷ | — | +¶¹⁷⁸ | +¹⁷⁹ | +¹⁸⁰ | — | +¹⁸¹ | +‡¹⁸² |
| ↓ Atrial fibrillation | — | — | +¶¹⁸³ | x¹⁸⁴ | +¹⁸⁵ | — | — | — |
| ↓ Cancer | +¹⁸⁶ | +¶¹⁸⁷ | +¶¹⁸⁸ | x¹⁸⁹ | — | — | +‡¹⁹⁰ | +‡¹⁹¹ |

+Positive association
crConflicting results
—No information found
na No association
xNegative association
*Peroxisome proliferator-activated receptor
†Low dose aspirin
‡Proposed
§Known to lower blood pressure
¶By inference, as opposite agent causes opposite action
#Possibility raised
**Further studies needed
††Study of fructose-fed rats
§§Study of growing rabbits
¶¶Plus COX-2 inhibitor

As evidenced by the data represented in Tables 2 and 3, preventative factors (i.e., pharmaceutical and lifestyle agents) are considered to operate by a single opposing mechanism involving parasympathetic activation. Multiple major and diverse preventative agents for IHD, hypertension, and diabetes exhibit findings which are opposite those of risk factors for IHD, hypertension, and diabetes, and this information also provides evidence that preventative agents operate by a single mechanism.

Evidence for parasympathetic activation by preventative factors is based on findings that multiple and diverse pharmaceutical and lifestyle agents express a decrease in sympathetic activity and an increase in parasympathetic activity; also, findings of preventative factors are directly opposite those of sympathetic fight/fight and risk factors.

While Tables 2 and 3 are not designed to provide full evidence for the idea that any preventative factor can oppose any risk factor, the risk factors of endothelial dysfunction, a tendency to thrombosis, dyslipidemia, inflammation, and insulin resistance are improved by multiple pharmaceutical and lifestyle agents. However, the findings that pharmaceutical and lifestyle preventative agents rather uniformly express parasympathetic activation and attributes of parasympathetic activation provides strong evidence that preventative factors operate by one mechanism—and thus would oppose and completely or partially counteract any risk factor, including the risk attributable to artificial blood. As additional evidence that pharmaceutical and lifestyle preventative agents operate by one mechanism, the various agents generally were shown to have the same actions of improving cognitive function and bone density, and decrease the incidence of Alzheimer's disease, atrial fibrillation, and cancer.

Thus, based on a synthesis of the data from the literature, which is uniquely reflected in Tables 1-3, there is unexpected close similarity of findings of multiple and diverse risk factors, and multiple and diverse preventative factors; these similarities are considered to offer strong evidence that risk and preventative factors operate by a single opposing mechanism and through a risk/prevention balance.

As artificial blood basically represents a risk factor, the principle of overbalancing risk factors by added preventative factors should improve the risk/prevention balance and allow for significant reduction of the risk of CV events from administration of artificial blood. Without wishing to be bound by any particular theory, there are likely two different types of risk factors that lead to adverse CV events. First are risk factors that cause general sympathetic activation, with concomitant expression of the four attributes of sympathetic activation, i.e., endothelial dysfunction expressing thrombosis/vasoconstriction, inflammation, dyslipidemia, and insulin resistance. Risk factors that lead to general sympathetic activation include, for example, emotional stress and cold exposure. Second are risk factors that activate only one of the four sympathetic attributes but lead to a general sympathetic activation of the autonomic nervous system through the expression of the other three attributes. One sympathetic attribute influences the others because of the highly integrated nature of the autonomic nervous system. Examples of the second type of risk factor are diet-induced dyslipidemia and infection-induced inflammation. It can be seen in Table 1 that dyslipidemia and inflammation are associated with sympathetic activation and lead to the activation of the other three corresponding attributes of sympathetic activation.

For CV events, the critical factor is the expression of thrombosis/vasoconstriction by risk factors; CV events generally are attributed to thromboses, and has been attributed to spasm (Hellstrom Med Hypotheses 2003; 60:36-51). Because of this, the adverse CV risk of artificial blood is considered to be due to an overbalance of thrombosis/vasoconstriction, which can be opposed by the anti-thrombosis/vasodilative forces of the preventative measures of the invention.

The tendency of artificial blood to cause CV events is regarded as a result of the toxicity of hemoglobin molecules which seep into the walls of blood vessels and cause inflammation, resulting in spasm and clotting and consequent myocardial infarction. The preventative agents described herein would reduce the incidence of infarcts, based on anti-thrombosis/vasodilation and anti-inflammatory properties.

There are two preferred drugs for prevention of cardiovascular events from artificial blood, i.e., a statin, and an ACE inhibitor; it is agreed that ACE inhibitors are more effective than angiotensin receptor blockers in preventing infarction. See, e.g., Tsuyuki R T, McDonald M A. Circulation 2006; 114:855-860. Other agents known to prevent infarctions as peroxisome proliferator-activated receptor (PPAR) agents, and vasodilators also can be considered to increase the level of protection. Aspirin can be added if excessive bleeding is not a concern.

As the risk of artificial blood has been related to vascular inflammation, anti-inflammatory corticosteroids might reduce the incidence of cardiovascular events due to artificial blood. It was found that very low dose of inhaled corticosteroids may reduce the risk of acute myocardial infarctions (Huiart L et al. Eur Respir J 2005; 25:634). Also favorably, if myocardial infarction might occur, the use of even high doses of intravenous corticosteroids should not be harmful; high intravenous doses of methylprednisolone had no adverse myocardial effects given early in the course of clinical myocardial infarction (Peters R W et al. Chest 1978; 73:483).

In yet another embodiment of the present invention, the methods and compositions, and programs of this invention are directed at individuals who are in need of therapy with artificial blood, and who are at an elevated CV risk prior to the initiation of therapy with a first pharmaceutical agent, where the individuals who are at elevated CV risk include, but are not limited to, those with systemic lupus erythematosus; diabetes; angina pectoris; manifest coronary artery disease; stroke; hypertension; hypercholesterolemia; kidney disease; Chlamydia infection; Bartonella infection; obstructive pulmonary disease; who are on hemodialysis; who have received an organ transplant; who are obese; who are elderly; who have a family history of heart disease, atherosclerosis, or stroke; who are or have been cigarette smokers; or who have a history of myocardial infarction, transient ischemic attacks, stroke, atherosclerosis, stress, dyslipidemia, endothelial dysfunction, or peripheral vascular disease.

According to the present invention, preventative measures act against the artificial blood and also against standard risk factors; in each case, the risk/prevention balance is improved.

### Artificial Blood

It is well understood that artificial blood products may prove to be quite useful in surgical and trauma patient situations. See, e.g., Gould, S. A., et al., The Life-Sustaining Capacity of Human Polymerized Hemoglobin When Red Blood Cells Might Be Unavailable, Journal of the American College of Surgeons, 195, 445-455 (2002); Gould, S. A., et al., Clinical Utility of Human Polymerized Hemoglobin as a Blood Substitute After Acute Trauma and Urgent Surgery, The Journal of Trauma, 43, 325-331 (1997). The U.S. Food and Drug Administration has yet to approve an artificial blood product for human use. Past clinical trials have been linked to increases in hypertension, heart attacks, and other serious adverse cardiovascular events. See, e.g., Burton, T. M., FDA to Weigh Using Fake Blood in Trauma Trial, Wall Street J., Jul. 6, 2006, at B1.

The method of manufacture of, and the artificial blood product POLYHEME® have been described in U.S. Pat. No. 4,826,811, which is incorporated by reference herein in its entirety. POLYHEME®, a derivative of human hemoglobin, is currently the subject of an Investigational New Drug Application that is under review by the U.S. Food and Drug Administration. See Grant of Interim Extension of the Term of U.S. Pat. No. 4,826,811, 71 Fed. Reg. 119 (Jun. 21, 2006).

Further, the artificial blood product HEMOPURE®, derived from bovine hemoglobin, has been approved for human use in South Africa and has undergone significant study in the United States. See, e.g., Jahr, J. S., et al, Pivotal Phase III Study: Safety of Polymerized Bovine Hemoglobin (HBOC-201, HEMOPURE®) as Compared to RBC in Patients Undergoing Orthopedic Surgery, available from the U.S. Food and Drug Administration. Increased CV events are associated with the administration of HEMOPURE®. See id. See also U.S. Patent Appln. Publication No. 2005/0143565A1, which is incorporated herein by reference in its entirety.

Additional examples of artificial blood properties and methods of manufacture are well known in the art. See, e.g., U.S. Patent Appln. Publication No. 2006/0014671; U.S. Pat. No. 6,552,173; U.S. Pat. No. 5,691,452; U.S. Pat. No. 5,194,590, which are incorporated by reference herein in their entirety.

### Hydroxymethylglutaryl-Coenzyme A (HMG-CoA) Reductase Inhibitors (“Statins”)

HMG-CoA (3-hydroxy-3-methylglutaryl-coenzyme A) reductase is the enzyme which catalyzes the rate limiting step of cholesterol biosynthesis. HMG-CoA reductase inhibitors, also known as statins, are molecules which inhibit the enzymatic activity of HMG-CoA reductase and have been used to treat patients suffering from hypercholesterolemia. The first such inhibitor (compactin or Mevastatin) was isolated in 1976 (Endo, A. et al. F.E.B.S. Lett., 72: 323-326, 1976) and since then many other natural and chemically modified versions of Mevastatin have been identified and developed for clinical use.

Currently available statins include lovastatin, simvastatin, pravastatin, fluvastatin, cerivastatin and atorvastatin. Lovastatin (disclosed in U.S. Pat. No. 4,231,938) and simvastatin (ZOCOR®; disclosed in U.S. Pat. No. 4,444,784 and WO 00/53566) are administered in the lactone form. After absorption, the lactone ring is opened in the liver by chemical or enzymatic hydrolysis, and the active hydroxy acid is generated. Pravastatin (PRAVACHOL®; disclosed in U.S. Pat. No. 4,346,227) is administered as the sodium salt. Fluvastatin (LESCOL®; disclosed in U.S. Pat. No. 4,739,073), atorvastatin calcium salt (LIPITOR®; see U.S. Pat. No. 5,273,995) and cerivastatin sodium salt (also known as rivastatin; see U.S. Pat. No. 5,177,080) are also well known statins.

Recent studies have shown that, in addition to treatment of hyperlipidemia, HMG-CoA reductase inhibitors are useful in the treatment of acne and/or skin aging (see, e.g. Breton, L. et al. U.S. Pat. No. 5,902,805); can increase nitric oxide (NO)-mediated vasodilation and blood vessel relaxation (see e.g., Liao, J. K. et al. WO 99/18952); and can help prevent a second or additional myocardial infarction (see, e.g., Behounek, B. D. et al. U.S. Pat. No. 5,674,893; Olukotun, A. Y. et al. U.S. Pat. No. 5,622,985).

Behounek, B. D. et al. (U.S. Pat. No. 5,674,893) have shown that patients with one or more risk factors for a coronary event such as hypercholesterolemia, who are treated with an HMG-CoA reductase inhibitor, such as pravastatin, experience a rapid marked and significant reduction in adverse CV events. Thus, although a certain number of patients having one or more risk factors for coronary events are expected to suffer an adverse CV event, such as a myocardial infarction and/or unstable angina, it has unexpectedly been found that such patients when treated with an HMG-CoA reductase inhibitor, such as pravastatin, have a rapid and sizable reduction in such adverse CV events. What is even more remarkable is the fact that such reduction in adverse CV events occur within one year and usually within 6 months of treatment and even sooner. This is especially significant inasmuch as until now it has been the generally held view that a treatment effect on cardiac event rates appears only after a lag phase of 2 years, as seen in the Coronary Primary Prevention Trial (JAMA 1984; 251:351-364) and the Helsinki Heart Study (N. Engl. J. Med. 1987; 317:1237-1245).

According to the Prospective Study of Pravastatin in the Elderly at Risk (PROSPER) study, the HMG Co-A reductase inhibitor pravastatin was shown to reduce the risk of CV events (15% reduction versus placebo) in those with existing (secondary prevention) and in those at high risk of developing (primary prevention) vascular disease (Shepherd, J et al. The Lancet 360:1623 (2002)).

The terms “cholesterol-lowering agent,” “cholesterol-lowering drug” and “lipid-lowering agent” as used herein refer to a pharmacologically active, pharmaceutically acceptable agent that, when administered to a human subject, has the effect of modifying serum cholesterol levels. More particularly, the cholesterol-lowering agent lowers serum low density lipoprotein (LDL) cholesterol levels, or inhibits oxidation of LDL cholesterol, whereas high density lipoprotein (HDL) serum cholesterol levels may be lowered, remain the same, or be increased.

As used herein, the terms “hydroxymethylglutaryl coenzyme A (HMG-CoA) reductase inhibitors” and “statins” are synonymous and refer to members of a class of compounds that inhibit 3-hydroxy-3-methylglutaryl coenzyme A (HMG-CoA) reductase. The HMG-CoA reductase inhibitors belong to the broader class of lipid lowering agents. This enzyme catalyzes the conversion of HMG-CoA to mevalonate, which is an early and rate-limiting step in the biosynthesis of cholesterol. Examples of HMG-CoA reductase inhibitors that may be used include but are not limited to lovastatin (MEVACOR®; see U.S. Pat. No. 4,231,938), simvastatin (ZOCOR®; see U.S. Pat. No. 4,444,784), pravastatin (PRAVACHOL®; see U.S. Pat. No. 4,346,227), fluvastatin (LESCOL®; see U.S. Pat. No. 5,354,772), atorvastatin (LIPITOR®; see U.S. Pat. No. 5,273,995), cerivastatin (also called rivastatin; see U.S. Pat. No. 5,177,080), mevastatin (see U.S. Pat. No. 3,883,140), fluindostatin (Sandoz XU-62-320), velostatin (also called synvinolin; see U.S. Pat. Nos. 4,448,784 and 4,450,171), and compounds related to these as described in the cited references. Some other examples of HMG-CoA reductase inhibitors that may be used are, without limitation, presented in U.S. Pat. No. 6,264,938 at Table 1 and U.S. Pat. No. 5,622,985, columns 3 through 6. All pharmaceutically acceptable HMG-CoA reductase inhibitors are included in this invention.

In a specific embodiment, the statins of the present invention are atorvastatin, fluvastatin, lovastatin, pravastatin, rosuvastatin, and simvastatin, and especially atorvastatin.

Compounds that inhibit the activity of HMG-CoA reductase can be readily identified by using assays well known in the art; see, as examples, the assays described or cited in U.S. Pat. No. 4,231,938 at column 6, and in International Patent Publication WO 84/02131 at pp. 30-33.

### The Renin-Angiotensin System (RAS) and the Anti-Angiotensins

The renin-angiotensin system (RAS) is an endocrine cascade which traditionally has been thought to regulate blood volume, vascular tone, and Na homeostasis. Angiotensin-converting enzyme (ACE), the final enzyme in the cascade, is the key catalytic step in production of the peptide hormone angiotensin II (Ang II). The enzyme cleaves two amino acids from the inactive prohormone angiotensin I (Ang I) to form the biologically active octapeptide Ang II, a potent vasoconstrictor. In addition, ACE inactivates bradykinin (BK) in a two-step cleavage process first to the inactive peptide BK 1-7 followed by further cleavage to BK 1-5 (Stewart, Handbook of Inflammation, Volume 6: Mediators of the Inflammatory Process, pp. 189-217, Elsevier Science Publishers (1989)). ACE is also capable of hydrolyzing other peptides, including some neuropeptides such as gonadotropin-releasing hormone (Skigdel and Erdos, J. Clin. and Exper. Theory and Practice 1997; A9 (2&3):243-259).

The RAS has recently been implicated as an important adverse pathogenic mechanism in CV disease (Dzau, Circulation 1988; 77:4-13). As stated above, ACE plays a key role in the RAS. The enzyme converts Ang I to Ang II and also hydrolyzes vasodilator and antiproliferative kinins such as BK. The Ang II produced by the action of ACE also has powerful non-vasoconstrictive effects. Ang II can promote proliferation of myocardial and vascular smooth muscle and cause neointimal hyperplasia after arterial wall injury (Lindpaintner et al. J. Cardiovasc. Pharmacol. 1992; 20:S41-S47). Although Ang I can be converted to Ang II in the absence of ACE (Kinoshita et al. J. Biol. Chem. 1991; 266:19192-19197, Urata et al. J. Clin. Invest. 1993; 91:1269-1281, Urata et al. Circ. Res. 1990; 66:883-890), administration of an ACE inhibitor substantially diminishes the proliferative and pressor effects of Ang I, suggesting that ACE-mediated production of Ang II is an important physiologic process (Swales and Dzau, Am. Heart J. 1992; 123:1412-1413). The RAS has been implicated in CV disease primarily because of its role in fluid volume and blood pressure control, but the growth promoting effects of Ang II on smooth muscle and myocardium may also be directly involved in disease processes. In addition, increased ACE-mediated hydrolysis of BK, a potent local vasodilator, may have adverse effects. The RAS has also been implicated in left ventricular remodeling after myocardial infarction. This results in progressive left ventricular dilation and contractile dysfunction (J. Cardiovasc. Pharmacol. 1992; 20:S41-S47).

The terms “anti-angiotensin agent” and “inhibitor of the renin-angiotensin system” as used herein are synonymous and refer to a pharmacologically active, pharmaceutically acceptable agent that inhibits, directly or indirectly, the adverse effects of angiotensin, particularly angiotensin II. Included, without limitation, are agents that: inhibit angiotensin II synthesis; inhibit angiotensin II binding to the AT₁ receptor; or inhibit renin activity.

### Angiotensin-Converting Enzyme (ACE) Inhibitors

ACE inhibitors have major roles as vasodilators in hypertension and CHF and are among the most efficient drugs for treating these disorders (see, e.g., Opie et al. “Angiotensin Converting Enzyme Inhibitors and Conventional Vasodilators,” in Lionel H. Opie, Drugs for the Heart, Third Edition, W B Saunders Company, 1991, p 106). Several clinical trials indicate that ACE inhibitors prolong survival in a broad spectrum of patients with myocardial infarction and heart failure, ranging from those who are asymptomatic with ventricular dysfunction to those who have symptomatic heart failure but are normotensive and hemodynamically stable. For example, one study demonstrated a 40% reduction in mortality at 6 months in patients with severe heart failure (The CONSENSUS Trial Study Group, N. Engl. J. Med. 1987; 316:1429; The CONSENSUS Trial Study Group, N. Engl. J. Med. 1991; 325:293).

The benefits of treatment are not restricted to survival. The addition of an ACE inhibitor to diuretic therapy improves the control of heart failure, an important symptomatic benefit. This reduces the need for hospitalization and probably improves the patient's quality of life. There may also be economic benefits for the health care system. Since their introduction in the mid-1980s, angiotensin converting enzyme (ACE) inhibitors have become well established for the treatment of hypertension and heart failure.

Selection of the patients to be treated is not based on the presence or absence of altered ACE levels or the presence of any of the polymorphisms in the gene, however, but solely on the observation of symptoms in which the known vasodilator properties of the ACE inhibitors have been proven to be useful. These patients are typically treated with relatively low doses of the ACE inhibitors in an amount effective to decrease blood pressure.

Recent studies have shown that the use of ACE inhibitors in patients with myocardial infarction has improved survival and reduced the rates of non-fatal CV events, especially when these agents are used for long term treatment in high risk patients such as those with signs of heart failure, evidence of left ventricular systolic dysfunction, or both (Pfeffer, M A. et al. New Engl. J. Med. 2003; 349:1893).

The Heart Outcomes Prevention Evaluation (HOPE) study investigated whether the addition of the ACE inhibitor ramipril to the current medical regimen of high-risk patients with diabetes mellitus could lower risks of adverse CV events. The rate of the combined primary outcome of myocardial infarction, stroke, and cardiovascular death was significantly lower in the ramipril group compared to the placebo group (relative risk reduction was 25%) (See, The Lancet 2000; 355:253).

The term “angiotensin converting enzyme (ACE) inhibitors” as used herein refers to any compound that inhibits the conversion of angiotensin I to angiotensin II. Because angiotensin I has only about 1% of the hypertensive activity of angiotensin II, ACE inhibitors are generally effective in reducing blood pressure and the other adverse CV effects caused by angiotensin II. ACE has numerous substrates other than angiotensin I, including bradykinin. By interfering with the conversion of bradykinin, ACE inhibitors increase bradykinin levels; this mechanism may contribute to the efficacy of ACE inhibitors.

Numerous ACE inhibitors have been synthesized. Most of these compounds can be classified into three groups based on their chemical structure: (1) sulfhydryl—(also called mercapto-) containing ACE inhibitors, including captopril and agents that are structurally related to captopril, such as fentiapril, pivalopril, zofenopril and alacepril; (2) dicarboxyl-containing ACE inhibitors, including enalapril and agents that are structurally related to enalapril, such as lisinopril, benazepril, quinapril, moexipril, ramipril, spirapril, perindopril, indolapril, pentopril, indalapril and cilazapril; and (3) phosphorus-containing ACE inhibitors, structurally related to fosinopril. Many of the ACE inhibitors are esters developed for high oral bioavailability, but with low potency in themselves; they must be converted to particular metabolites in the body that have potent activity.

ACE inhibitors are well known in the art, and the use of any pharmaceutically acceptable ACE inhibitor, including any of those mentioned in the preceding paragraph, is included in this invention, including mixtures thereof and/or their pharmaceutically acceptable salts. Some further examples of ACE inhibitors that may be used in the practice of this invention are, without limitation, AB-103, ancovenin, benazeprilat, BRL-36378, BW-A575C, CGS13928C, CL242817, CV-5975, Equaten, EU-4865, EU-4867, EU-5476, foroxymithine, FPL 66564, FR-900456, Hoe-065, 15B2, indolapril, ketomethylureas, KRI-1177, KRI-1230, L681176, libenzapril, MCD, MDL-27088, MDL-27467A, moveltipril, MS-41, nicotianamine, pentopril, phenacein, pivopril, rentiapril, RG-5975, RG-6134, RG-6207, RGH0399, ROO-911, RS-10085-197, RS-2039, RS 5139, RS-86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y23785, Yissum, P-0154, zabicipril, Asahi Brewery AB-47, alatriopril, BMS182657, Asahi Chemical C-111, Asahi Chemical C-112, Dainippon DU-1777, mixanpril, Prentyl, zofenoprilat, 1(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-66564, idrapril, perindoprilat, Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, trandolapril, trandolaprilat, ceranapril, moexipril, quinaprilat, spirapril, and those listed in U.S. Pat. No. 6,248,729.

In a specific embodiment, the ACE inhibitors are benazepril, captopril, cilazapril, delapril, enalapril, fentiapril, fosinopril, indolapril, lisinopril, moexipril, perindopril, pivopril, quinapril, ramipril, spirapril, trandolapril, and zofenopril; especially captopril, enalapril, fosinopril, lisinopril, quinapril, ramipril, and trandolapril. An exemplary ACE inhibitor is ramipril.

### Salts, Prodrugs, and Metabolites

Any of the foregoing active agents may be administered in the form of a salt, ester, amide, prodrug, active metabolite or the like, provided that the salt, ester, amide, prodrug, or active metabolite is pharmaceutically acceptable and pharmacologically active in the present context. Salts, esters, amides, prodrugs or metabolites of the active agents may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a drug in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

### Formulations and Routes of Administration

The artificial blood is administered parenterally. The preventative pharmaceutical agent may be administered orally, intranasally, rectally, sublingually, buccally, parenterally, or transdermally. Dosage forms may include tablets, trochees, capsules, caplets, dragees, lozenges, parenterals, liquids, powders, and formulations designed for implantation or administration to the surface of the skin. In general, it is expected that oral dosage forms of the preventative pharmaceutical agent will be the most convenient. All dosage forms may be prepared using methods that are standard in the art (see e.g., Remington's Pharmaceutical Sciences, 16th ed. A. Oslo. ed., Easton, Pa. (1980), and all later editions, e.g. 17th, 18th, and 19th editions).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

In a specific embodiment, the present invention provides for a pharmaceutical composition, wherein the artificial blood and one or more of the foregoing pharmaceutical agents for reducing or prevent the risk of CV events are in a parenteral dosage form.

It is preferred to introduce the preventative agent(s) promptly to ensure prompt protection. If possible, as with elective surgery, preventative pharmaceutical agents should be given before the artificial blood is given to have the protective agents in place and operative prior to transfusion; in this circumstance, preventative pharmaceutical agents can be given orally or parentally. In emergency situations, when pretreatment is not possible, preventative pharmaceutical agents should be given parentally, as by IV, as soon as possible; in non emergent situations, oral dosage usually would be preferable. Preventative pharmaceutical agents should be continued for at least a week after the last unit of artificial blood is infused, as it is known that HEMOPURE® persists in the plasma for 5-7 days (Senior K., Mol. Med. Today. 1998 April; 4(4):139.). Generally, preventative pharmaceutical agents should be continued until after the time span of known post transfusion infarctions.

### Dosages

With respect to preventative agents, it is expected that the skilled practitioner will adjust dosages on a case-by-case basis using methods well established in clinical medicine. Dosage of preventative agents should include consideration of loss of preventative agents through continuing hemorrhage. Nevertheless, the following general guidelines with respect to the artificial blood, statins, ACE inhibitors, and ARBs are provided.

Of the two preferred pharmaceutical preventative agents (statins and ACE inhibitors), statins should be used at a high approved dose. According to the PDR, fluvastatin (LESCOL®) is usually prescribed in unit dosages of 20 and 40 mg with 80 mg extended release dosages available. Lovastatin (ALTOCOR®), Mevacor is usually prescribed in unit dosages of 10, 20 and 40 mg with extended release 10, 20, 40 and 60 mg. Pravastatin (PRAVACHOL®) is usually prescribed in unit dosages of 10, 20, 40 and 80 mg. Simvastatin (ZOCOR®) is usually prescribed in unit dosages of 5, 10, 20, 40, and 80 mg. Atorvastatin (LIPITOR®) is usually prescribed in unit dosages of 10, 20, 40 and 80 mg. Rosuvastatin (CRESTOR®) is usually prescribed in unit dosages of 5, 10, and 40 mg. Here, the preferred statin is atorvastatin at a dose of either 40 or 80 mg a day, with 80 mg preferable. There is evidence that 80 mg of atorvastatin prevented stroke (N Engl J Med 2006; 355:549-559), improved acute coronary syndromes better than standard doses of pravastatin (J Am Coll Cardiol 2005; 46:1405-1410), improved endothelium-dependent vasodilation better than 10 mg (Am J Cardiol 2005; 96:1361-1364), was more beneficial than 10 mg in stable coronary disease (N Engl J Med 2005; 352:1425-1435), and reduced inflammatory markers better (Expert Opin Pharmacother 2005; 6:915-927). High-dose atorvastatin (80 mg) reduced clinical events in cases of acute coronary syndromes better than standard dosages (Ray K K et al. Early and late benefits of high-dose atorvastatin in patients with acute coronary syndromes: results from the PROVE IT-TIMI 22 trial, J Am Coll Cardiol 2005; 46:1405-1410). Further, high-dose atorvastatin (80 mg) improved endothelium-dependent vasodilation better than 10 mg (van der Harst P, et al. Effect of intensive versus moderate lipid lowering on endothelial function and vascular responsiveness to angiotensin II in stable coronary artery disease, Am J Cardiol 2005; 96:1361-1364). It has been determined that high-dose atorvastatin (80 mg) is more beneficial in improving stable angina than 10 mg (LaRosa J C et al. Intensive lipid lowering with atorvastatin in patients with stable coronary disease. N Engl J Med 2005; 352:1425-1435). Additionally, high-dose atorvastatin (80 mg) is associated with a greater reduction in inflammatory markers (Ray K K, Atorvastatin and cardiovascular protection: a review and comparison of recent clinical trials, Expert Opin Pharmacother 2005; 915;915-927).

In the case of ACE inhibitors, specific dosages are, for example, from about 1 mg to about 800 mg, more particularly from about 5 mg to about 600 mg, and especially from about 10 mg to about 400 mg.

For example, preferred dosages, according to the PDR are: from about 5 mg to about 20 mg, preferably 5 mg, 10 mg or 20 mg of benazepril; from about 6 mg to about 100 mg, preferably 6.25 mg, 12.5 mg, 25 mg, 50 mg, 75 mg or 100 mg of captopril; from about 2 mg to about 20 mg, preferably 2.5 mg, 5 mg, 10 mg or 20 mg of enalapril; from about 10 mg to about 20 mg, preferably 10 mg or 20 mg of fosinopril; from about 2 mg to about 4 mg, preferably 2 mg or 4 mg of perindopril; from about 5 mg to about 20 mg, preferably 5 mg, 10 mg or 20 mg of quinapril; or from about 1 mg to about 5 mg, preferably 1.25 mg, 2.5 mg, or 5 mg of ramipril.

The daily dosage may be provided in either a single dose or multiple dose regimen, with the latter being generally preferred. These are guidelines since the actual dose must be carefully selected and titrated by the attending physician based upon clinical factors unique to each patient. The optimal daily dose will be determined by methods known in the art and will be influenced by factors such as the age of the patient, the disease state, side effects associated with the particular agent being administered and other clinically relevant factors. In some cases, a patient may already be taking medications at the time that treatment with the present combination is initiated. These other medications may or may not be continued, depending upon the judgment of the attending physician.

With regard to aspirin, the dose of 325 mg/day is preferred, as the dose of 160 to 325 mg/day is recommended for acute treatment (Awry E H, Loscalzo J. Aspirin. Circulation 2000; 101; 1206-1218). Aspirin is contraindicated in situations with severe bleeding.

Hydrocortisone is not one of the basic group of preferred preventative agents (statins and ACE inhibitors), but may nevertheless be used within the judgment of the skilled artisan. Doses of oral hydrocortisone range from 20 to 240 mg per day, and between 20 and 40 mg is regarded as an appropriate dose. If hydrocortisone is used, it should not be abruptly terminated, but terminated in a step-wise fashion. The hydrocortisone can be used either orally or parenterally.

### Drug Combinations

Since two or more preventative pharmaceutical agents may be used together in a combination therapy, preferably a statin and ACE inhibitor, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts.

As pharmaceutical preventative agents are generally additive, a combination therapy such as a statin and ACE inhibitor is preferred. Further, where bleeding is controlled, consideration should be given to utilizing such a combination therapy further including aspirin.

In a further embodiment of the present invention is a dosage regimen for individuals who are at elevated risk of seriously adverse CV events and who are already taking a preventative pharmaceutical agent, such as a statin or an ACE inhibitor, to reduce this risk. A consideration of these factors is well within the purview of the ordinarily skilled clinician. For example, the clinician can determine an effective dose of any additional or supplemental dosage of a statin or ACE inhibitor that may be necessary. Alternatively, if a patient is already on a regimen that includes a statin and there is a risk of side effects from an increase of statin dosage, a clinician may decide to co-administer the artificial blood with an effective dosage of an ACE inhibitor and/or aspirin.

Without further elaboration, it is believed that one skilled in the art can, using the preceding description, utilize the present invention to its fullest extent. The following examples are, therefore, to be construed as merely illustrative and not limiting to the remainder of the disclosure in any way whatsoever.

### EXAMPLES

### Example 1

### Dosage Regimen for Co-Administration of the Artificial Blood Product HEMOPURE® with the Statin Atorvastatin (LIPITOR®)

Table 4 provides dosage regimens for the administration of the artificial blood product HEMOPURE® with LIPITOR® in a surgical context. As illustrated in Table 4, the dosage ranges of HEMOPURE® to LIPITOR® may vary. The exact dose should be chosen based on the patient's condition and the patient's risk of a seriously adverse CV event (e.g., myocardial infarction). For example, a patient in a surgical situation that has a high risk of infarct prior to artificial blood therapy is administered a higher ratio of LIPITOR® to HEMOPURE®. A patient who has a low risk of infarct prior to administration of artificial blood is administered a lower dose of LIPITOR®.

TABLE 4

| Medical | HEMOPURE ® | |
|---|---|---|
| Condition | Dosing | LIPITOR ® Dosing |
| Elective | Up to 300 g over | Up to 80 mg per day |
| orthopedic | 6 days, before, | beginning before, during or |
| surgery | during or after | after surgery, continuing |
| | surgery | for 7 days or more after |
| | | the Hemopure dosing |
| | | regimen is complete |
| Non-Cardiac | Up to 210 g over | |
| elective | 6 days before, | |
| surgery | during or after | |
| | surgery | |
| Post- | Up to 120 g over | |
| cardiopulmonary | 3 days post-surgery | |
| bypass surgery | | |
| Aortic aneurism | Up to 150 g over | |
| reconstruction | 4 days, first dose | |
| surgery | administered | |
| | during or after | |
| | surgery | |

### Example 2

### Daily Dosage Regimen for Administration of the Artificial Blood Product POLYHEME® with the Statin Atorvastatin (LIPITOR®)

Table 5 provides dosage regimens for the administration of the artificial blood product POLYHEMEL® with LIPITOR® in a trauma context. As illustrated in Table 4, the dosage ranges of POLYHEME® to LIPITOR® may vary. The exact dose should be chosen based on the patient's condition and the patient's risk of a seriously adverse CV event (e.g., myocardial infarction), if known. For example, a patient in a trauma situation that has a high risk of infarct prior to artificial blood therapy is administered a higher ratio of LIPITOR® to POLYHEME®. A patient who has a low risk of infarct prior to administration of artificial blood is administered a lower dose of LIPITOR®.

TABLE 5

| Medical | POLYHEME ® | |
|---|---|---|
| Condition | Dosing | LIPITOR ® Dosing |
| Acute trauma | Up to 1000 g | Up to 80 mg per day beginning |
| | | before, during or after artificial |
| | | blood dosing, continuing for 7 days |
| | | or more after the Polyheme dosing |
| | | regimen is complete |

The present invention is not limited in scope by the specific embodiments described herein. Indeed, various modifications of the invention in addition to those described herein will become apparent to those skilled in the art from the foregoing description. Such modifications are intended to fall within the scope of the appended claims. It is further to be understood that all values given in the foregoing examples are approximate, and are provided for purposes of illustration.

Patents, patent applications, publications, product descriptions, and protocols which are cited throughout this application are incorporated herein by reference in their entireties for all purposes.

### REFERENCE LIST FOR TABLES 1-3

1. Lefkowitz R J, Hoffman B B, Taylor P. Neurotransmission: the autonomic and somatic motor nervous systems. In: Hardman J G, Limbird L E, Molinoff P B, Ruddon R W, Gilman A G, editors. The Pharmacological Basis of Therapeutics. New York: McGraw-Hill, 1996: 105-139.
2. Hijmering M L, Stroes E S G, Olijhoek J et al. Sympathetic activation markedly reduces endothelium-dependent, flow-mediated vasodilation. J Am Coll Cardiol. 2002; 39:683-688.
3. Cannon W B. Bodily Changes in Pain, Hunger, Fear and Rage. An Account of Recent Researches into the Function of Emotional Excitement. 2nd ed. Boston: Charles T. Branford Co, 1953.
4. Black P H. Stress and the inflammatory response: a review of neurogenic inflammation. Brain Behav Immun. 2002; 16:622-653.
5. Remme W J. The sympathetic nervous system and ischaemic heart disease. Eur Heart J. 1998; 19 (suppl F):F62-F71.
6. Julius S, Majahalme S. The changing face of sympathetic overactivity in hypertension. Ann Med. 2000; 32:365-370.
7. Esler M, Rumantir M, Wiesner G et al. Sympathetic nervous system and insulin resistance: from obesity to diabetes. Am J Hypertens. 2001; 14:304 S-309S.
8. Verma S, Anderson T J. Fundamentals of endothelial function for the clinical cardiologist. Circulation. 2002; 105:546-549.
9. Taccarino G, Ciccarelli M, Sorriento D et al. AKT participates in endothelial dysfunction in hypertension. Circulation. 2004; 109:2587-2593.
10. Expert Panel on Detection EaToHBC. Detection, evaluation, and treatment of high blood cholesterol in adults (Adult Treatment Panel III): third report of the National Cholesterol Education Program (NCEP) Expert Panel. Circulation. 2002; 106:3145-3421.
11. Chobanian A V, Bakris G L, Black H R et al. The Seventh Report of the Joint National Committee on Prevention, Detection, Evaluation, and Treatment of High Blood Pressure: the JNC 7 report. JAMA. 2003; 289:2560-2572.
12. Welzig C M, Shin D G, Park H J et al. Lipid Lowering by Pravastatin Increases Parasympathetic Modulation of heart rate: Gαᵢ₂, a Possible Marker for Parasmpathetic Responsiveness. Circulation. 2003; 108:2743-2746.
13. De Curtis A, D'Adamo M C, Amore C et al. Experimental arterial thrombosis in genetically or diet induced hyperlipidemia in rats—role of vitamin K-dependent clotting factors and prevention by low-intensity oral anticoagulation. Thromb Haemost. 2001; 86:1440-1448.
14. Hutter R, Valdiviezo C, Sauter B V et al. Caspase-3 and tissue factor expression in lipid-rich plaque macrophages: evidence for apoptosis as link between inflammation and atherothrombosis. Circulation. 2004; 109:2001-2008.
15. Reaven G M, Lithell H, Landsberg L. Hypertension and associated metabolic abnormalities—the role of insulin resistance and the sympathoadrenal system. N Engl J Med. 1996; 334:374-381.
16. Ridker P M, Glynn R J, Hennekens C H. C-reactive protein adds to the predictive value of total and HDL cholesterol in determining risk of first myocardial infarction. Circulation. 1998; 97:2007-2011.
17. Sesso H D, Buring J E, Rifai N et al. C-reactive protein and the risk of developing hypertension. JAMA. 2003; 290:2945-2951.
18. Pickup J C. Inflammation and activated innate immunity in the pathogenesis of type 2 diabetes. Diabetes Care. 2004; 27:813-823.
19. Esmon C T. Crosstalk between inflammation and thrombosis. Maturitas. 2004; 47:305-314.
20. Ross R. Atherosclerosis—an inflammatory disease. N Engl J Med. 1999; 340:115-126.
21. Grundy S M. Inflammation, hypertension, and the metabolic syndrome. JAMA. 2003; 290:3000-3002.
22. Julius S, Gudbrandsson T. Early association of sympathetic overactivity, hypertension, insulin resistance, and coronary risk. J Cardiovasc Pharmacol. 1992; 20 (suppl 8):S40-S48.
23. Boden G, Hoeldtke R D. Nerves, fat, and insulin resistance. N Engl J. Med. 2003; 349: 1966-1967.
24. Sobel B E. Insulin resistance and thrombosis: a cardiologist's view. Am J. Cardiol. 1999; 84:37J-41J.
25. Morrow L A, Halter J B. Diabetes in the older adult. Geriatrics. 1988; 43 (suppl):57-65.
26. Esler M, Hastings J, Lambert G et al. The influence of aging on the human sympathetic nervous system and brain norepinephrine turnover. Am J Physiol. 2002; 282:R909-R916.
27. May O, Arildsen H, Møller M. Parasympathetic function during deep breathing in the general population: relation to coronary risk factors and normal range. J Intern Med. 1999; 245:287-294.
28. Wilkerson W R, Sane D C. Aging and thrombosis. Semin Thromb Hemost. 2002; 28:555-567.
29. Gostynski M, Gutzwiller F, Kuulasmaa K et al. Analysis of the relationship between total cholesterol, age, body mass index among males and females in the WHO MONICA Project. Int J Obes Relat Metab Dis. 2004; 28:1082-1090.
30. Franceschi C, Bonafe M, Valensin S et al. Inflamm-aging: an evolutionary perspective on immunosenescence. Ann N Y Acad Sci. 2000; 908:244-254.
31. Røder M E, Schwartz R S, Prigeon R L et al. Reduced pancreatic B cell compensation to the insulin resistance of aging: impact on proinsulin and insulin levels. J Clin Endocrinol Metab. 2000; 85:2275-2280.
32. Paradis G, Lambert M, O'Loughlin J et al. Blood pressure and adiposity in children and adolescents. Circulation. 2004; 110: 1832-1838.
33. Eckel R H, Barouch W W, Ershow A G. Report of the National Heart, Lung, and Blood Institute-National Institute of Diabetes and Digestive and Kidney Diseases Working Group on the pathophysiology of obesity-associated CV disease. Circulation. 2002; 105:2923.
34. Alvarez G E, Beske S D, Ballard T P et al. Sympathetic neural activation in visceral obesity. Circulation. 2002; 106:2533.
35. Pathak A, Smih F, Galinier M et al. Insulin downregulates M(2)-muscarinic receptors in adult rat atrial cardiomyocytes: a link between obesity and cardiovascular complications. Int J Obes Relat Metab Discord. 2005; 29:176-182.
36. Berg A H, Scherer P E. Adipose tissue, inflammation, and CV disease. [Review][133 refs]. Circ Res. 2005; 96:939-949.
37. Barengo N C, Hu G, Kastarinen M et al. Low physical activity as a predictor for antihypertensive drug treatment in 25-64-year-old populations in eastern and south-western Finland. [see comment]. J Hypertens. 2005; 23:293-299.
38. Sullivan P W, Morrato E H, Ghushchyan V et al. Obesity, inactivity, and the prevalence of diabetes and diabetes-related cardiovascular comorbidities in the U.S., 2000-2002. Diabetes Care. 2005; 28:1599-1603.
39. Hellstrom H R. Evidence in support of the spasm of resistance vessel concept of ischemic heart disease: an update in 1993. Med. Hypotheses. 1993; 41:11-22.
40. Goldsmith R L, Bigger J T Jr, Steinman R C et al. Comparison of 24-hour parasympathetic activity in endurance-trained and untrained young men. J Am Coll Cardiol. 1992; 20:552-558.
41. Lee K W, Lip G Y H. Effects of lifestyle on hemostasis, fibrinolysis, and platelet reactivity: a systematic review. Arch Intern Med. 2003; 163:2368-2392.
42. Das U N. Anti-inflammatory nature of exercise. Nutrition. 2004; 20:323-326.
43. LaMonte M J, Blair S N, Church T S. Physical activity and diabetes prevention. [Review][84 refs]. J Appl Physiol. 2005; 99: 1205-1213.
44. Rozanski A, Blumenthal J A, Kaplan J. Impact of psychological factors on the pathogenesis of CV disease and implications for therapy. Circulation. 1999; 99:2192-2217.
45. Yan L L, Liu K, Matthews K A et al. Psychosocial factors and risk of hypertension: the Coronary Artery Risk Development in Young Adults (CARDIA) study. JAMA. 2003; 290:2138-2148.
46. Rosmond R. Stress induced disturbances of the HPA axis: a pathway to Type 2 diabetes? Med Sci Monit. 2003; 9:RA35-RA39.
47. Black P H, Garbutt L D. Stress, inflammation and CV disease. J Psychosom Res. 2002; 52:1-23.
48. Mezzacappa E S, Kelsey R M, Katkin E S et al. Vagal rebound and recovery from psychological stress. Psychosom Med. 2001; 63:650-657.
49. Black P H. The inflammatory response is an integral part of the stress response: implications for atherosclerosis, insulin resistance, type II diabetes and metabolic syndrome X. Brain Behav Immun. 2003; 17:350-364.
50. Haire-Joshu D, Glascow R E, Tibbs T L. Smoking and diabetes. Diabetes Care. 1999; 22:1887-1898.
51. Hjemdahl P. Smoking, nicotine and thrombotic risk—a role for platelet dependent thrombin generation? Eur Heart J. 2001; 22:16-18.
52. Tsiara S, Elisaf M, Mikhailidis D P. Influence of smoking on predictors of vascular disease. Angiology. 2003; 54:507-530.
53. Eliasson B. Cigarette smoking and diabetes. Prog Cardiovasc Dis. 2003; 45:405-413.
54. Manfredini R, Gallerani M, Portaluppi F et al. Relationships of the circadian rhythms of thrombotic, ischemic, hemorrhagic, and arrhythmic events to blood pressure rhythms. Ann N Y Acad Sci. 1996; 783:141-158.
55. la Fleur S E, Kalsbeek A, Wortel J et al. A daily rhythm in glucose tolerance: a role for the suprachiasmatic nucleus. Diabetes. 2001; 50:1237-1243.
56. Trotti R, Rondanelli M, Cuzzoni G et al. Circadian temporal organization of lipidic fractions in elderly people: entrainment to the dietary schedule. Aging Clin Exp Res. 2002; 14:94-99.
57. Panzer S E, Dodge A M, Kelly E A B et al. Circadian variation of sputum inflammatory cells in mild asthma. J Allergy Clin Immunol. 2003; 111:308-312.
58. Spencer F A, Goldberg R J, Becker R C et al. Seasonal distribution of acute myocardial infarction in the second National Registry of Myocardial Infarction. J Am Coll Cardiol. 1998; 31:1226-1233.
59. Sun Z, Wang X, Wood C E et al. Genetic AT_{1A} receptor deficiency attenuates cold-induced hypertension. Am Journal Physiol. 2005; 288:R433-R439.
60. Tseng C L, Brimacombe M, Xie M et al. Seasonal patterns in monthly hemoglobin A1c values. Am J Epidemiology. 2005; 161:565-574.
61. Kawahara J, Sano H, Fukuzaki H et al. Acute effects of exposure to cold on blood pressure, platelet function and sympathetic nervous activity in humans. Am J. Hypertens. 1989; 2:724-726.
62. Gyllerup S, Lanke J, Lindholm L H et al. Cold climate is an important factor in explaining regional differences in coronary mortality even if serum cholesterol and other established risk factors are taken into account. Scot Med J. 1993; 38:169-172.
63. Mercer J B, Østerud B, Tveita T. The effect of short-term cold exposure on risk factors for CV disease. Thromb Res. 1999; 95:93-104.
64. Torsoni M A, Carvalheira J B, Pereira-Da-Silva M et al. Molecular and functional resistance to insulin in hypothalamus of rats exposed to cold. Am J Physiol. 2003; 285:E216-E223.
65. Hayden M, Pignone M, Phillips C et al. Aspirin for the primary prevention of cardiovascular events: a summary of the evidence for the U.S. Preventive Services Task Force. Ann Intern Med. 2002; 136:161-172.
66. Pfeffer M A, McMurray J J V, Velazquez E J et al. Valsartan, captopril, or both in myocardial infarction complicated by heart failure, left ventricular dysfunction, or both. N Engl J Med. 2003; 349:1893-1906.
67. Chiquette E, Ramirez G, DeFronzo R. A meta-analysis comparing the effect of thiazolidinediones on CV risk factors. Arch Intern Med. 2004; 164:2097-2104.
68. Michels K B, Manson J E. Postmenopausal hormone therapy: a reversal of fortune. Circulation. 2003; 107:1830-1833.
69. Bybee K A, Kopecky S L, Williams B A et al. Reduced creatine kinase release with statin use at the time of myocardial infarction. Int J. Cardiol. 2004; 96:461-466.
70. Brouwer M A, van den Bergh P J P C, Aengevaeren W R M et al. Aspirin plus coumarin versus aspirin alone in the prevention of reocclusion after fibrinolysis for acute myocardial infarction: results of the Antithrombotics in the Prevention of Reocclusion In Coronary Thrombolysis (APRICOT)-2 Trial. Circulation. 2002; 106:659-665.
71. Takagi T, Akasaka T, Yamamuro A et al. Troglitazone reduces neointimal tissue proliferation after coronary stent implantation in patients with non-insulin dependent diabetes mellitus: a serial intravascular ultrasound study. J Am Coll Cardiol. 2000; 36:1529-1535.
72. Golomb B A, Ritchie J B, Criqui M H et al. Statins lower blood pressure: results from the UCSD statin study. Circulation. 2004; 110 (suppl III): III-402.
73. Wald N J, Law M R. A strategy to reduce CV disease by more than 80%. BMJ. 2003; 326:1419-1422.
74. Dobrian A D, Schriver S D, Khraibi A A et al. Pioglitazone prevents hypertension and reduces oxidative stress in diet-induced obesity. Hypertension. 2004; 43:48-56.
75. Wyss J M, Carlson S H. Effects of hormone replacement therapy on the sympathetic nervous system and blood pressure. Curr Hypertens Rep. 2003; 5:241-246.
76. Magen E, Viskoper R, Mishal J et al. Resistant arteral hypertension and hyperlipidemia: atorvastatin, not vitamin C, for blood pressure control. Isr Med Assoc J. 2004; 6:742-746.
77. Hermida R C, Ayala D E, Calvo C et al. Administration time-dependent effects of aspirin on blood pressure in untreated hypertensive patients. Hypertension. 2003; 41:1259-1267.
78. Freeman D J, Norrie J, Sattar N et al. Pravastatin and the development of diabetes mellitus: evidence for a protective treatment effect in the West of Scotland Coronary Prevention Study. Circulation. 2001; 103:357-362.
79. Scheen A J. Prevention of type 2 diabetes mellitus through inhibition of the renin-angiotensin system. Drugs. 2004; 64:2537-2565.
80. Buchanan T A, Xiang A H, Peters R K et al. Preservation of pancreatic □-cell function and prevention of type 2 diabetes by pharmacological treatment of insulin resistance in high-risk hispanic women. Diabetes. 2002; 51:2796-2803.
81. Kanaya A M, Herrington D, Vittinghoff E et al. Glycemic effects of postmenopausal hormone therapy: the Heart and Estrogen/progestin Replacement Study: a randomized, double-blind, placebo-controlled trial. Ann Int Med. 2003; 138:1-9.
82. Paniagua J A, López-Miranda J, Escribano A et al. Cerivastatin improves insulin sensitivity and insulin secretion in early-state obese type 2 diabetes. Diabetes. 2002; 51:2596-2603.
83. The Heart Outcomes Prevention Evaluation Study Investigators. Effects of an angiotensin-converting-enzyme inhibitor, ramipril, on cardiovascular events in high-risk patients. N Engl J. Med. 2000; 342: 145-153.
84. Yki-Jarvinen H. Thiazolidinediones. N Eng J. Med. 2004; 351:1106-1118.
85. Abelson K S P, Kommalage M, Höglund U. Spinal cholinergic involvement after treatment with aspirin and paracetamol in rats. Neurosci Lett. 2004; 368:116-120.
86. Kontopoulos A G, Athyros V G, Papageorgiou A A et al. Effect of quinapril or metoprolol on circadian sympathetic and parasympathetic modulation after acute myocardial infarction. Am J. Cardiol. 1999; 84: 1164-1169.
87. Manhem K, Brandin L, Ghanoum B et al. Acute effects of transdermal estrogen on hemodynamic and vascular reactivity in elderly postmenopausal healthy women. J Hypertens. 2003; 21:387-394.
88. Kishi T, Hirooka Y, Mukai Y et al. Atorvastatin causes depressor and sympatho-inhibitory effects with upregulation of nitric oxide synthases in stroke-prone spontaneously hypertensive rats. J Hypertens. 2003; 21:379-386.
89. Nagai Y, Sugiyama Y, Nakatsumi Y et al. Rapid amelioration of muscle sympathetic nerve activity by pioglitazone in an obese Type 2 diabetic patient. Diabet Med. 2003; 20:868-869.
90. Vongpatanasin W, Tuncel M, Mansour Y et al. Transdermal estrogen replacement therapy decreases sympathetic activity in postmenopausal women. Circulation. 2001; 103:2903-2908.
91. Quyyumi A A. Effects of aspirin on endothelial dysfunction in atherosclerosis. Am J Cardiol. 1998; 82:31 S-33S.
92. Sidhu J S, Cowan D, Kaski J-C. The effects of rosiglitazone, a peroxisome proliferator-activated receptor-gamma agonist, on markers of endothelial cell activation, C-reactive protein, and fibrinogen levels in non-diabetic coronary artery disease patients. J Am Coll Cardiol. 2003; 42:1757-1763.
93. Rosenson R S, Tangney C C. Antiatherothrombotic properties of statins: implications for cardiovascular event reduction. JAMA. 1998; 279:1643-1650.
94. Chiang N, Bermudez E A, Ridker P M et al. Aspirin triggers antiinflammatory 15-epi-lipoxin A₄ and inhibits thromboxane in a randomized human trial. PNAS. 2004; 101:15178-15183.
95. Gryglewski R J, Swies J, Uracz W et al. Mechanisms of angiotensin-converting enzyme inhibitor induced thrombolysis in Wistar rats. Thromb Res. 2003; 110:323-329.
96. Rosendaal F R, Helmerhorst F M, Vandenbroucke J P. Female hormones and thrombosis. Arterioscler Thromb Vasc Biol. 2002; 22:201-210.
97. Hundal R S, Petersen K F, Mayerson A B et al. Mechanism by which high-dose aspirin improves glucose metabolism in type 2 diabetes. J Clin Invest. 2002; 109:1321-1326.
98. Kon V, Jabs K. Angiotensin in atherosclerosis. Curr Opin Nephrol Hypertens. 2004; 13:291-297.
99. Khan M, Xu Y, Edwards G et al. Effects of pioglitazone on the components of diabetic dyslipidaemia: results of double-blind, multicentre, randomised studies. Int J Clin Pract. 2004; 58:907-912.
100. Faludi A A, Aldrighi J M, Bertolami M C et al. Progesterone abolishes estrogen and/or atorvastatin endothelium dependent vasodilatory effects. Atherosclerosis. 2004; 177:89-96.
101. Lefer D J. Statins as potent antiinflammatory drugs. Circulation. 2002; 106:2041-2042.
102. Hsuch W A, Lyon C J, Quinones M J. Insulin resistance and the endothelium. Am J. Med. 2004; 117:109-117.
103. Thomas T N, Rhodin J A, Clark L et al. A comparison of the anti-inflammatory activities of conjugated estrogens and 17-□ estradiol. Inflamm Res. 2003; 52:452-460.
104. Greenfield J R, Samaras K, Jenkins A B et al. Moderate alcohol consumption, estrogen replacement therapy, and physical activity are associated with increased insulin sensitivity: is abdominal adiposity the mediator? Diabetes Care. 2003; 26:2734-2740.
105. Starr J M, McGurn B, Whiteman M et al. Life long changes in cognitive ability are associated with prescribed medications in old age. Int J Geriatr Psychiatry. 2004; 327-332.
106. Shepherd C E, Piguet O, Broe G A et al. Histocompatibility antigens, aspirin use and cognitive performance in non-demented elderly subjects. J Neuroimmunol. 2004; 148:178-182.
107. Tedesco M A, Ratti G, Di Salvo G et al. Does the angiotensin II receptor antagonist losartan improve cognitive function? Drugs Aging. 2002; 19:723-732.
108. Fillit H M, Butler R N, O'Connell A W et al. Achieving and maintaining cognitive vitality with aging. Mayo Clin Proc. 2002; 77:681-696.
109. Lupattelli G, Scarponi A M, Vaudo G et al. Simvastatin increases bone mineral density in hypercholesterolemic postmenopausal women. Metabolism. 2004; 53:744-748.
110. Carbone L D, Tylavsky F A, Cauley J A et al. Association between bone mineral density and the use of nonsteroidal anti-inflammatory drugs and aspirin: impact of cyclooxygenase selectivity. J Bone Miner Res. 2003; 18:1795-1802.
111. Hamerman D. Osteoporosis and atherosclerosis: biological linkages and the emergence of dual-purpose therapies. Q J. Med. 2005; 98:467-484.
112. Clemons M, Goss P. Estrogen and the risk of breast cancer. N Engl J. Med. 2001; 344:276-285.
113. Rea T D, Breitner J C, Psaty B M et al. Statin use and the risk of incident dementia. Arch Neurol. 2005; 62:1047-1051.
114. Broe G A, Grayson D A, Creasey H M et al. Anti-inflammatory drugs protect against Alzheimer disease at low doses. Arch Neurol. 2000; 57:1586-1591.
115. Combs C K, Johnson D E, Karlo J C et al. Inflammatory mechanisms in Alzheimer's disease: inhibition of beta-amyloid-stimulated proinflammatory responses and neurotoxicity by PPARgamma agonists. J Neurosci. 2000; 20:558-567.
116. Zandi P P, Carlson M C, Plassman B L et al. Hormone replacement therapy and incidence of Alzheimer Disease in older women: the Cache County Study. JAMA. 2002; 288:2123-2129.
117. Young-Xu Y, Jabbour S, Goldberg R et al. Usefulness of statin drugs in protecting against atrial fibrillation in patients with coronary artery disease. Am J. Cardiol. 2003; 92:1379-1383.
118. Healey J S, Baranchuk A, Crystal E et al. Prevention of atrial fibrillation with angiotensin-converting enzyme inhibitors and angiotensin receptor blockers: a meta-analysis. J Am Coll Cardiol. 2005; 45:1832-1839.
119. Poynter J N, Gruber S B, Higgins P D R et al. Statins and the risk of colorectal cancer. N Engl J Med. 2005; 352:2184-2192.
120. Gonzalez-Perez A, Garcia Rodriguez L A, Lopez-Ridaura R. Effects of non-steroidal anti-inflammatory drugs on cancer sites other than the colon and rectum: a meta-analysis. BMC Cancer. 2003; 3:28.
121. Lindberg H, Nielsen D, Jensen B V et al. Angiotensin converting enzyme inhibitors for cancer treatment? Acta Oncol. 2004; 43:142-152.
122. Nahlé Z. PPAR trilogy from metabolism to cancer. Curr Opin Clin Nutr Metab Care. 2004; 7:397-402.
123. Thompson P D, Buchner D, Piña I L et al. Exercise and physical activity in the prevention and treatment of atherosclerotic CV disease: a statement from the Council on Clinical Cardiology (Subcommittee on Exercise, Rehabilitation, and Prevention) and the Council on Nutrition, Physical Activity, and Metabolism (Subcommittee on Physical Activity). Circulation. 2003; 107:3109-3116.
124. de Lorgeril M, Salen P. Is alcohol anti-inflammatory in the context of coronary heart disease? Heart. 2004; 90:355-357.
125. He K, Song Y, Daviglus M L et al. Accumulated evidence on fish consumption and coronary heart disease mortality: a meta-analysis of cohort studies. Circulation. 2004; 109:2705-2711.
126. Ros E, Núñez I, Pérez-Heras A et al. A walnut diet improves endothelial function in hypercholesterolemic subjects: a randomized crossover trial. Circulation. 2004; 109:1609-1614.
127. de Lorgeril M, Salen P, Martin J-L et al. Mediterranean diet, traditional risk factors, and the rate of cardiovascular complications after myocardial infarction: final report of the Lyon Diet Heart Study. Circulation. 1999; 99:779-785.
128. Sies H, Schewe T, Heiss C et al. Cocoa polyphenols and inflammatory mediators. Am J Clin Nutr. 2005; 81 (suppl):304S-312S.
129. GISSI-Prevenzione Investigators. Dietary supplementation with n-3 polyunsaturated fatty acids and vitamin E after myocardial infarction: results of the GISSI-Prevenzione trial. Lancet. 1999; 354:447-455.
130. Franco V, Oparil S, Carretero O A. Hypertensive therapy: part II. Circulation. 2004; 109:3081-3088.
131. Matthews K A, Katholi C R, McCreath H et al. Blood pressure reactivity to psychological stress predicts hypertension in the CARDIA study. Circulation. 2004; 110:74-78.
132. Psaltopoulou T, Naska A, Orfanos P et al. Olive oil, the Mediterranean diet, and arterial blood pressure: the Greek European Prospective Investigation into Cancer and Nutrition (EPIC) study. Am J Clin Nutr. 2004; 80:1012-1018.
133. Al-Awwadi N A, Bornet A, Azay J et al. Red wine polyphenols alone or in association with ethanol prevent hypertension, cardiac hypertrophy, and production of reactive oxygen species in the insulin-resistant fructose-fed rat. J Agric Food Chem. 2004; 52:5593-5597.
134. Krantz D S, McCeney M K. Effects of psychological and social factors on organic disease: a critical assessment of research on coronary heart disease. Annu Rev Psychol. 2002; 53:341-369.
135. Geleijnse J M, Giltay E J, Grobbee D E et al. Blood pressure response to fish oil supplementation: metaregression analysis of randomized trials. J Hypertens. 1920; 1493-1499.
136. Sjöström L, Lindroos A-K, Peltonen M et al. Lifestyle, diabetes, and cardiovascular risk factors 10 years after bariatric surgery. N Eng J. Med. 2004; 351:2683-2693.
137. Wales J K. Does psychological stress cause diabetes? Diabet Med. 1995; 12:109-112.
138. Howard A A, Arnsten J H, Gourevitch M N. Effect of alcohol consumption on diabetes mellitus: a systematic review. Ann Intern Med. 2004; 140:211-219.
139. Jiang R, Manson J E, Stampfer M J et al. Nut and peanut butter consumption and risk of type 2 diabetes in women. JAMA. 2002; 288:2554-2560.
140. Biesalski H K. Diabetes preventive components in the Mediterranean diet. Eur J. Nutr. 2004; 43 (suppl 1):i26-i30.
141. Inui A, Kitaoka H, Majima M et al. Effect of the Kobe earthquake on stress and glycemic control in patients with diabetes mellitus. Arch Intern Med. 1998; 158:274-278.
142. Goldsmith R L, Bloomfield D M, Rosenwinkel E T. Exercise and autonomic function. Coron Artery Dis. 2000; 11:129-135.
143. Laaksonen D E, Laitinen T, Schönberg J et al. Weight loss and weight maintenance, ambulatory blood pressure and cardiac autonomic tone in obese persons with the metabolic syndrome. J Hypertens. 2003; 21:371-378.
144. Sakakibara M, Takeuchi S, Hayano J. Effect of relaxation training on cardiac parasympathetic tone. Psychophysiology. 1994; 31:223-228.
145. Murata K, Araki S, Yokoyama K et al. Autonomic neurotoxicity of alcohol assessed by heart rate variability. J Auton Nerv Syst. 1994; 48:105-111.
146. Das U N. Beneficial effect(s) of n-3 fatty acids in CV diseases: but, why and how? Prostaglandins Leukot Essent Fatty Acids. 2000; 63:351-362.
147. Trombetta I C, Batalha L T, Rondon M U P B et al. Weight loss improves neurovascular and muscle metaboreflex control in obesity. Am J Physiol. 2003; 285:H974-H982.
148. Lucini D, Covacci G, Milani R et al. A controlled study of the effects of mental relaxation on autonomic excitatory responses in healthy subjects. Psychosom Med. 1997; 59:541-552.
149. van de Borne P, Mark A L, Montano N et al. Effects of alcohol on sympathetic activity, hemodynamics, and chemoreflex sensitivity. Hypertension. 1997; 29:1278-1283.
150. Jennings G L R, Chin-Dusting J P F, Kingwell B A et al. Modulation of vascular function by diet and exercise. Clin Exp Hypertens. 1997; 19:727-737.
151. Higashi Y, Sasaki S, Kurisu S et al. Regular aerobic exercise augments endothelium-dependent vascular relaxation in normotensive as well as hypertensive subjects: role of endothelium-derived nitric oxide. Circulation. 1999; 100:1194-1202.
152. Ziccardi P, Nappo F, Giugliano G et al. Reduction of inflammatory cytokine concentrations and improvement of endothelial functions in obese women after weight loss over one year. Circulation. 2002; 105:804-809.
153. Spieker L E, Hürlimann D, Ruschitzka F et al. Mental stress induces prolonged endothelial dysfunction via endothelin-A receptors. Circulation. 2002; 105:2817-2820.
154. Teragawa H, Fukuda Y, Matsuda K et al. Effect of alcohol consumption on endothelial function in men with coronary artery disease. Atherosclerosis. 2002; 165:145-152.
155. Esposito K, MARBella R, Ciotola M et al. Effect of a Mediterranean-style diet on endothelial dysfunction and markers of vascular inflammation in the metabolic syndrome. JAMA. 2004; 292:1440-1446.
156. Lacoste L, Hung J, Lam J Y T. Acute and delayed antithrombotic effects of alcohol in humans. Am J Cardiol. 2001; 87:82-85.
157. Mruk J S, Webster M W I, Heras M et al. Flavone-8-acetic acid (flavonoid) profoundly reduces platelet-dependent thrombosis and vasoconstriction after deep arterial injury In vivo. Circulation. 2000; 101:324-328.
158. Klein S, Burke L E, Bray G A et al. Clinical implications of obesity with specific focus on cardiovascular disease. A statement for professional from the American Heart Association Council on Nutrition, Physical Activity, and Metabolism. Circulation. 2004; 110:2952-2967.
159. Wannamethee S G, Lowe G D O, Shaper G et al. The effects of different alcoholic drinks on lipids, insulin and haemostatic and inflammatory markers in older men. Thromb Haemost. 2003; 90:1080-1087.
160. Tapsell L C, Gillen L J, Patch C S et al. Including walnuts in a low-fat/modified-fat diet improves HDL cholesterol-to-total cholesterol ratios in patients with type 2 diabetes. Diabetes Care. 2004; 27:2777-2783.
161. Chiba H, Uehara M, Wu J et al. Hesperidin, a citrus flavonoid, inhibits bone loss and decreases serum and hepatic lipids in ovariectomized mice. J Nutr. 2003; 133:1892-1897.
162. Kasapis C, Thompson P D. The effects of physical activity on serum C-reactive protein and inflammatory markers: a systematic review. J Am Coll Cardiol. 2005; 45:1563-1569.
163. Lutgendorf S, Logan H, Kirchner H L et al. Effects of relaxation and stress on the capsaicin-induced local inflammatory response. Psychosom Med. 2000; 62:524-534.
164. Albert M A, Glynn R J, Ridker P M. Alcohol consumption and plasma concentration of C-reactive protein. Circulation. 2003; 107:443-447.
165. Raikkonen K, Keltikangas-Jarvinen L, Adlercreutz H et al. Psychosocial stress and the insulin resistance syndrome. Metabolism. 1996; 45:1533-1538.
166. Sirtori C R, Galli C. N-3 fatty acids and diabetes. Biomed Pharmacother. 2002; 56:397-406.
167. Garcia-Lorda P, Megias R, I, Salas-Salvado J. Nut consumption, body weight and insulin resistance. Eur J Clinl Nutr. 2003; 57 (suppl 1):S8-S11.
168. Weuve J, Kang J H, Manson J E et al. Physical activity, including walking, and cognitive function in older women. JAMA. 2004; 292:1454-1461.
169. Bagger Y Z, Tanko L B, Alexandersen P et al. The implications of body fat mass and fat distribution for cognitive function in elderly women. Obes Res. 2004; 12:1519-1526.
170. Stampfer M J, Kang J H, Chen J et al. Effects of moderate alcohol consumption on cognitive function in women. N Engl J. Med. 2005; 352:245-253.
171. Kalmijn S. Fatty acid intake and the risk of dementia and cognitive decline: a review of clinical and epidemiological studies. J Nutr Health Aging. 2000; 4:202-207.
172. Joseph J A, Shukitt-Hale B, Casadesus G. Reversing the deleterious effects of aging on neuronal communication and behavior: beneficial properties of fruit polyphenolic compounds. Am J Clin Nutr. 2005; 81 (suppl):313S-316S.
173. MacKelvie K J, Petit M A, Khan K M et al. Bone mass and structure are enhanced following a 2-year randomized controlled trial of exercise in prepubertal boys. Bone. 2004; 34:755-764.
174. Guney E, Kisakol G, Ozgen G et al. Effect of weight loss on bone metabolism: comparison of vertical banded gastroplasty and medical intervention. Obes Surg. 2003; 13:383-388.
175. Bakhireva L N, Barrett-Connor E, Kritz-Silverstein D et al. Modifiable predictors of bone loss in older men: a prospective study. Am J Prev Med. 2004; 26:436-442.
176. Judex S, Wohl G R, Wolff R B et al. Dietary fish oil supplementation adversely affects cortical bone morphology and biomechanics in growing rabbits. Calcif Tissue Int. 2000; 66:443-448.
177. Abbott R D, White L R, Ross G W et al. Walking and dementia in physically capable elderly men. JAMA. 2004; 292:1447-1453.
178. Wilson R S, Evans D A, Bienias J L et al. Proneness to psychological distress is associated with risk of Alzheimer's disease. Neurology. 2003; 61: 1479-1485.
179. Luchsinger J A, Tang M X, Siddiqui M et al. Alcohol intake and risk of dementia. J Am Geriatr Soc. 2004; 52:540-546.
180. Morris M C, Evans D A, Bienias J L et al. Consumption of fish and n-3 fatty acids and risk of incident Alzheimer disease. Arch Neurol. 2003; 60:940-946.
181. Panza F, Solfrizzi V, Colacicco A M et al. Mediterranean diet and cognitive decline. Public Health Nutr. 2004; 7:959-963.
182. Weinreb O, Mandel S, Amit T et al. Neurological mechanisms of green tea polyphenols in Alzheimer's and Parkinson's diseases. J Nutr Biochem. 2004; 15:506-516.
183. Houghton J L, Devlin C W, Besson W T I et al. Possible triggering of paroxysmal atrial fibrillation in normal hearts by psychological stressors: a report of two cases. Am J Med. Sci. 1990; 300:234-236.
184. Frost L, Vestergaard P. Alcohol and risk of atrial fibrillation or flutter: a cohort study. Arch Intern Med. 2004; 164: 1993-1998.
185. Mozaffarian D, Psaty B M, Rimm E B et al. Fish intake and risk of incident atrial fibrillation. Circulation. 2004; 110:368-373.
186. Alfano C M, Klesges R C, Murray D M et al. Physical activity in relation to all-site and lung cancer incidence and mortality in current and former smokers. Cancer Epidemiol Biomarkers Prev. 2004; 13:2233-2241.
187. Kuriyama S, Tsubono Y, Hozawa A et al. Obesity and risk of cancer in Japan. Int Journal Cancer. 2005; 113:148-157.
188. Reiche E M, Nunes S O, Morimoto H K. Stress, depression, the immune system, and cancer. Lancet Oncol. 2004; 5:617-625.
189. Bianchini F, Vainio H. Wine and resveratrol: mechanisms of cancer prevention? Eur J Cancer Prev. 2003; 12:417-425.
190. Visioli F, Grande S, Bogani P et al. The role of antioxidants in the Mediterranean diets: focus on cancer. Eur J Cancer Prev. 2004; 13:337-343.
191. Neuhouser M L. Dietary flavonoids and cancer risk: evidence from human population studies. Nutr Cancer. 2004; 50:1-7.

What is claimed is:

1. A method for reducing the risk of one or more adverse cardiovascular events in a subject to be treated with artificial blood, the method comprising administering:
(a) a therapeutically effective amount of artificial blood; and
(b) one or more preventative pharmaceutical agents in an amount effective to reduce the risk of one or more adverse cardiovascular events.

2. The method of claim 1, wherein a preventative pharmaceutical agents is selected from the group consisting of a statin and an ACE inhibitor.

3. The method of claim 2, wherein the preventative pharmaceutical agent is a statin.

4. The method of claim 1, wherein a preventative pharmaceutical agent is selected from the group consisting of aspirin, a corticosteroid, a PPAR agent, and a vasodilator.

5. The method of claim 1, wherein a preventative pharmaceutical agent is administered for at least one week.

6. The method of claim 1, wherein an adverse cardiovascular event is selected from the group consisting of myocardial infarction and stroke.

7. A method for reducing the risk of one or more adverse cardiovascular events in a subject to be treated with artificial blood, the method comprising co-administering:
(a) a therapeutically effective amount of artificial blood; and
(b) one or more preventative pharmaceutical agents in an amount effective to reduce the risk of one or more adverse cardiovascular events.

8. The method of claim 7, wherein artificial blood and a preventative pharmaceutical agent are co-administered parenterally in a unit dosage form.

9. The method of claim 7, wherein a preventative pharmaceutical agent is selected from the group consisting of a statin and an ACE inhibitor.

10. The method of claim 7, wherein a preventative pharmaceutical agent is selected from the group consisting of aspirin, a corticosteroid, a PPAR agent, and a vasodilator.

11. A kit for reducing the risk of one or more adverse cardiovascular events in a subject to be treated with artificial blood, comprising:
(a) a therapeutically effective amount of artificial blood; and
(b) instructions for administering one or more preventative pharmaceutical agents in an amount effective to reduce the risk of one or more adverse cardiovascular events.

12. The kit of claim 11, wherein a preventative pharmaceutical agent is selected from the group consisting of a statin and an ACE inhibitor.

13. The kit of claim 12, wherein the preventative pharmaceutical agent is a statin.

14. The kit of claim 11, wherein a preventative pharmaceutical agent is selected from the group consisting of aspirin, a corticosteroid, a PPAR agent, and a vasodilator.

15. The kit of claim 11, wherein the one or more preventative pharmaceutical agents comprise a statin and an ACE inhibitor.

16. The kit of claim 15, wherein the one or more preventative pharmaceutical agents additionally comprise aspirin and the subject is not at risk of severe bleeding.

17. A composition comprising:
(a) a therapeutically effective amount of artificial blood; and
(b) one or more preventative pharmaceutical agents in an amount effective to reduce the risk of at least one adverse cardiovascular event associated with administration of artificial blood.

18. The composition of claim 17, wherein a preventative pharmaceutical agent is selected from the group consisting of a statin and an ACE inhibitor.

19. The composition of claim 18, wherein the preventative pharmaceutical agent is a statin.

20. The composition of claim 17, wherein a preventative pharmaceutical agent is selected from the group consisting of aspirin, a corticosteroid, a PPAR agent, and a vasodilator.

21. A composition comprising:
(a) a therapeutically effective amount of artificial blood; and
(b) a statin in an amount effective to reduce the risk of an adverse cardiovascular event associated with administration of artificial blood.

22. The composition of claim 21, further comprising an ACE inhibitor.

23. The composition of claim 21, wherein the statin is atorvastatin.

24. The composition of claim 22, wherein the ACE inhibitor is ramipril.

25. The composition of claim 21, wherein the adverse cardiovascular event is selected from the group consisting of myocardial infarction and stroke.
